SEQUENCE LISTING (45 sequences)

SEQ ID NO: 1 - protein, 15 aa, Artificial Sequence
  Antennapedia Penetratin hydrophilic CPP
RQIKWFQNRRMKWKK

SEQ ID NO: 2 - protein, 19 aa, Artificial Sequence
  BMV Gag-(7-25) hydrophilic CPP
KMTRAQRRAAARRNRWTAR

SEQ ID NO: 3 - protein, 13 aa, Artificial Sequence
  D-Tat hydrophilic CPP
GRKKRRQRRRPPQ

SEQ ID NO: 4 - protein, 15 aa, Artificial Sequence
  FHV Coat-(35-49) hydrophilic CPP
RRRRNRTRRNRRRVR

SEQ ID NO: 5 - protein, 13 aa, Artificial Sequence
  HTLV-II Rex-(4-16) hydrophilic CPP
TRRQRTRRARRNR

SEQ ID NO: 6 - protein, 12 aa, Artificial Sequence
  PTD-4 hydrophilic CPP
PIRRRKKLRRLK

SEQ ID NO: 7 - protein, 12 aa, Artificial Sequence
  PTD-5 hydrophilic CPP
RRQRRTSKLMKR

SEQ ID NO: 8 - protein, 13 aa, Artificial Sequence
  R9-Tat hydrophilic CPP
GRRRRRRRRRPPQ

SEQ ID NO: 9 - protein, 18 aa, Artificial Sequence
  SynB1 hydrophilic CPP
RGGRLSYSRRRFSTSTGR

SEQ ID NO: 10 - protein, 10 aa, Artificial Sequence
  SynB3 hydrophilic CPP
RRLSYSRRRF

SEQ ID NO: 11 - protein, 11 aa, Artificial Sequence
  TAT hydrophilic CPP
YGRKKRRQRRR

SEQ ID NO: 12 - protein, 27 aa, Artificial Sequence
  Transportan amphipathic CPP
GWTLNSAGYLLGKINLKALAALAKKIL

SEQ ID NO: 13 - protein, 17 aa, Artificial Sequence
  MAP amphipathic CPP
KLALKLALKLALALKLA

SEQ ID NO: 14 - protein, 27 aa, Artificial Sequence
  SBP amphipathic CPP
MGLGLHLLVLAAALQGAWSQPKKKRKV

SEQ ID NO: 15 - protein, 27 aa, Artificial Sequence
  FBP amphipathic CPP
GALFLGWLGAAGSTMGAWSQPKKKRKV

SEQ ID NO: 16 - protein, 27 aa, Artificial Sequence
  MPG amphipathic CPP
GALFLGFLGAAGSTMGAWSQPKKKRKV

SEQ ID NO: 17 - protein, 27 aa, Artificial Sequence
  MPG(??NLS) amphipathic CPP
GALFLGFLGAAGSTMGAWSQPKSKRKV

SEQ ID NO: 18 - protein, 21 aa, Artificial Sequence
  Pep-1 amphipathic CPP
KETWWETWWTEWSQPKKKRKV

SEQ ID NO: 19 - protein, 21 aa, Artificial Sequence
  Pep-2 amphipathic CPP
KETWFETWFTEWSQPKKKRKV

SEQ ID NO: 20 - protein, 9 aa, Artificial Sequence
  nuclear localization domain of c-Myc
PAAKRVKLD

SEQ ID NO: 21 - protein, 25 aa, Artificial Sequence
  nuclear localization domain of EGL-13
MSRRRKANPTKLSENAKKLAKEVEN

SEQ ID NO: 22 - protein, 10 aa, Artificial Sequence
  nuclear localization domain of nucleoplasmin #1
PAATKKAGQA

SEQ ID NO: 23 - protein, 16 aa, Artificial Sequence
  nuclear localization domain of nucleoplasmin #2
KRPAATKKAGQAKKKK

SEQ ID NO: 24 - protein, 7 aa, Artificial Sequence
  nuclear localization domain of SV40
PKKKRKV

SEQ ID NO: 25 - protein, 29 aa, Artificial Sequence
  ER localization domain
MMSFVSLLLVGILFWATEAEQLTKCEVFQ

SEQ ID NO: 26 - protein, 25 aa, Artificial Sequence
  mitochondria localization domain
MLSLRQSIRFFKPATRTLCSSRYLL

SEQ ID NO: 27 - protein, 39 aa, Artificial Sequence
  chloroplast localization domain
MVAMAMASLQSSMSSLSLSSNSFLGQPLSPITLSPFLQG

SEQ ID NO: 28 - protein, 13 aa, Artificial Sequence
  peroxisome localization domain
  VARIANT 1: X represents any amino acid residue
  VARIANT 2: X represents any amino acid residue
  VARIANT 3: X represents any amino acid residue
  VARIANT 4: X represents any amino acid residue
  VARIANT 7: X represents any amino acid residue
  VARIANT 8: X represents any amino acid residue
  VARIANT 9: X represents any amino acid residue
  VARIANT 10: X represents any amino acid residue
  VARIANT 11: X represents any amino acid residue
XXXXRLXXXXXHL

SEQ ID NO: 29 - protein, 16 aa, Artificial Sequence
  vacuole localization domain #1
HSRFNPIRLPTTHEPA

SEQ ID NO: 30 - protein, 23 aa, Artificial Sequence
  vacuole localization domain #2
SSSSFADSNPIRPVTDRAASTLE

SEQ ID NO: 31 - protein, 15 aa, Artificial Sequence
  vacuole localization domain #3
VFAEAIAANSTLVAE

SEQ ID NO: 32 - protein, 8 aa, Artificial Sequence
  vacuole localization domain #4
NGLLVDTM

SEQ ID NO: 33 - protein, 22 aa, Artificial Sequence
  vacuole localization domain #5
VSGGVWDSSVETNATASLVSEM

SEQ ID NO: 34 - protein, 18 aa, Artificial Sequence
  vacuole localization domain #6
QAHPNFPLEMPGSDEVAK

SEQ ID NO: 35 - protein, 5 aa, Artificial Sequence
  Gly-rich linker #1
GGSSG

SEQ ID NO: 36 - protein, 6 aa, Artificial Sequence
  Gly-rich linker #2
GSGSGS

SEQ ID NO: 37 - protein, 11 aa, Artificial Sequence
  Gly-rich linker #3
GGSGGSGGSGG

SEQ ID NO: 38 - protein, 16 aa, Artificial Sequence
  Gly-rich linker #4
GGGGSLVPRGSGGGGS

SEQ ID NO: 39 - protein, 11 aa, Artificial Sequence
  Gly-rich linker #5
GGSGGHMGSGG

SEQ ID NO: 40 - protein, 8 aa, Artificial Sequence
  Gly-rich linker #6
SGGGSSHS

SEQ ID NO: 41 - protein, 10 aa, Artificial Sequence
  Gly-rich linker #7
SGGSGGSSHS

SEQ ID NO: 42 - protein, 13 aa, Artificial Sequence
  Gly-rich linker #8
SGGSGGSGGSSHS

SEQ ID NO: 43 - DNA, 49 bp, Artificial Sequence
  primer #1
ttagattagttcatggtcatagctgtttcctgttttcccagtcacgacg

SEQ ID NO: 44 - DNA, 24 bp, Artificial Sequence
  primer #2
cctgagcggataacaatttcacac

SEQ ID NO: 45 - DNA, 21 bp, Artificial Sequence
  Repeated sequence in the sequence to be circularized from Example 10
cctgtgtgaaattgttatccg

(19) United States
(12) Patent Application Publication
(10) Pub. No.: US 2023/0159939 A1
(43) Pub. Date: May 25, 2023

(54) METHODS FOR CIRCULARIZING LINEAR DOUBLE STRANDED NUCLEIC ACIDS

(71) Applicant: FABMID, EVRY-COURCOURONNES (FR)

(72) Inventor: Andrey PICHUGIN, Fresnes (FR)

(73) Assignee: FABMID, EVRY-COURCOURONNES (FR)

(21) Appl. No.: 17/920,575

(22) PCT Filed: Apr. 22, 2021
(86) PCT No.: PCT/EP2021/060603

§ 371 Date: Oct. 21, 2022

(30) Foreign Application Priority Data
Apr. 22, 2020 (EP) 20305391.3

(51) Int. Cl. C12N 15/64; C12N 9/00; C12N 9/12; C12N 9/22

(52) U.S. Cl. CPC C12N 15/64; C12N 9/93; C12N 9/1241; C12N 9/22; C12N 2800/10

(57) ABSTRACT

A method, in particular an in vitro method, for the circularization of a double stranded DNA nucleic acid. Also, a circularized double stranded DNA nucleic acid obtainable by the method. Another aspect pertains to a host cell comprising a circularized double stranded DNA nucleic acid obtainable by the method. Further, therapeutic and non-therapeutic uses of a circularized double stranded DNA nucleic acid obtainable by the method. Finally, a kit for the circularization of a double stranded DNA nucleic acid.

### FIELD OF INVENTION

The present invention relates to the field of circularization of linear double stranded nucleic acids, in particular linear double stranded (ds) DNA nucleic acids.

### BACKGROUND OF INVENTION

The field of nucleic acid engineering provides many applications, in particular for the purpose of human and veterinary therapy, such as the production of plasmids, notably for the production of recombinant proteins of therapeutic and/or economic interest, creation of DNA libraries for drug screening. These recombinant proteins may be used in turn as drugs to treat diseases. Illustratively, polypeptides of therapeutic and/or economic interest encompass, e.g., growth factors, antibodies, hormones, cytokines, enzymes, plasmatic factors, and the like.

For decades, tools have been intensively developed to assemble nucleic acids from various origins, which have been referred to as “molecular biology”. One of the main techniques of molecular biology consists in inserting a nucleic acid of interest into a circular nucleic acid vector, which facilitates nucleic acid cloning and amplification. For example, for human and veterinary medicine, human/animal gene encoding a protein of interest may be assembled within bacterial or viral nucleic acid vectors, so as to obtain hybrid circular recombinant nucleic acids enabling target expression of the human/animal protein within the human body. This technique relies upon the use of polymerase chain reaction (PCR), restriction enzymes, ligases, and often result in a poor yield, i.e. 10% at most, and hence often requires bacterial systems to amplify the final product to achieve higher yields.

Of importance, obtaining circular nucleic acids are one goal to be achieved, because circular nucleic acids are easier to handle, and more resistant to degradation, as compared to single stranded nucleic acids.

For illustrative purposes, one may cite Gibson et al. (Nature Methods, 2009, 6(5), 343-345), who disclosed a method for the one-step isothermal in vitro recombination of 2 ds linear DNA sharing terminal overlapping sequences. WO2017066943 disclosed a kit for in vitro assembly of a DNA fragment comprising a DNA endonuclease, a DNA exonuclease, a DNA polymerase and at least a DNA ligase.

Ellis et al. (Integr. Biol., 2011, 3, 109-118) have provided a comprehensive critical examination of DNA assembly strategies, based on the use of type II restriction enzymes (methods referred to BioBricks®, BglBricks, pairwise selection, Golden Gate), or on the use of overlapping (InFusion®, isothermal assembly, SLIC (Sequence and Ligase Independent Cloning).

Scientists have also been driving efforts into developing new tools in order to provide the state of the art with methods to self-assemble nucleic acids of interest, hence offering an alternative to the use of viral and/or bacterial vectors.

For example, EP2620497 discloses a method for producing a circular DNA from a single DNA molecule and which does not comprise circular DNA formed from multiple-molecule DNA. This method relies upon adding double stranded adapters to one extremity of the nucleic acid to be circularized.

WO2014079900 relates to a method for the in vitro production of supercoiled DNA minicircles having less than 250 base pairs, through a one-pot ligase-mediated circularization reaction of linear nicked double-stranded oligodeoxynucleotide in the presence of a DNA bending protein.

Chen Cheng et al. (J Med Genet 2019; 56:10-17) discloses a method for generating double stranded minicircles vector, which are bacteria-free. This method relies upon amplifying a nucleic acid of interest by PCR using primers introducing restrictions sites at each extremity of said nucleic acid. Digestion of the PCR product with the corresponding restriction enzyme and submitting the result of digestion to ligation allows obtaining circular nucleic acids.

There is a need to provide alternative methods for generating circularized double stranded nucleic acids. There is a need to provide high yield circularized double stranded nucleic acids, in particular without the requirement of using bacterial systems for amplification.

### SUMMARY

A first aspect of the invention relates to a method for the circularization of a double stranded DNA nucleic acid, said method comprising the steps of:
a) providing a linear or circular double stranded DNA nucleic acid comprising a nucleic acid of the following formula (I):

NHR1-SOR1ₓ-HR1-TBC-HR2-SOR2_{y}-NHR2 (I),

wherein:
TBC represents a core double stranded DNA nucleic acid to be circularized, in particular having a length of at least about 250 bp, preferably at least about 500 bp, more preferably at least about 1,000 bp;
HR1 and HR2 represent identical homologous double stranded DNA nucleic acids of identical orientation;
SOR1ₓ and SOR2_{y} represent nucleic acids having a length of about 5 bp to about 60 bp comprising a site of restriction capable of generating 3′ overhangs having a length of at least 4 nucleotides and having as 3′ terminal nucleotide A, T or G; and x and y are 0 or 1; when x and y are 1, TBC further comprises 2 sites of nicking restriction each located about 0 nucleotide to about 100 nucleotides respectively from the 3′ end of HR1 and from the 3′ end of HR2;
NHR1 and NHR2 represent distinct non-homologous double stranded DNA nucleic acids having a length of 0 bp to about 200 bp;
b1) when x and y are 0, digesting the circular double stranded DNA nucleic acid comprising a nucleic acid of formula (I) from step a), in the presence of a restriction enzyme that cleaves a restriction site that is not located in any one of HR1, HR2 and TBC, so as to obtain a linearized nucleic acid;
b2) when x and y are 1,
   optionally digesting the circular double stranded DNA nucleic acid comprising a nucleic acid of formula (I) from step a), in the presence of a restriction enzyme that cleaves a restriction site that is not located in any one of HR1, HR2 and TBC, so that to generate a linearized nucleic acid;
   digesting the circular, linear or linearized nucleic acid in the presence of (i) a polypeptide with nickase activity capable of introducing a nick within TBC, and (ii) restriction enzyme(s) capable of digesting the corresponding site of restriction in SOR1 and SOR2;
c) recessing both ends of identical orientation of the nucleic acid obtained in step b1) or step b2), in the presence of a polypeptide having a 3′-5′ nuclease activity, so that HR1 and HR2 are capable of forming overlapping overhangs;
d) annealing the DNA nucleic acid obtained at step c), thereby generating 2 gaps, 2 nicks, or 1 nick and 1 gap;
e) filling the 1 or 2 gaps generated at step d), step e) being optional when 2 nicks are generated at step d);
f) optionally removing the NHR1 and NHR2 at both 5′ ends, when the length of the NHR1 and NHR2 is superior to 0 bp;
g) sealing at least one nick, so as to obtain a circularized double stranded DNA nucleic acid, in which at least one strand is continuous.

In some embodiments, step b) or step c) is preceded by, or concomitantly performed with, a step comprising incubating the linear double stranded DNA nucleic acid of step a) with an alkaline phosphatase and/or a polypeptide with a type I exonuclease activity. In certain embodiments, step d) is performed in the presence of divalent and/or trivalent cations. In some embodiments, step e) is performed in the presence of a DNA polymerase with no strand displacement activity and with no 5′ to 3′ exonuclease activity and dNTPs and/or of oligonucleotides with 5′-phosphate having a length of about 8 nucleotides to about 100 nucleotides and being complementary to the nucleic acids of the gaps, provided the polypeptide having a 3′-5′ nuclease activity has been inactivated. In certain embodiments, step f) is performed in the presence of a polypeptide having a 5′-3′ nuclease activity, preferably Exonuclease VII (Exo VII), RecJ_{f} or Flap endonuclease 1 (FEN1). In some embodiments, step g) is performed in the presence of a mesophilic DNA ligase, preferably T4 DNA ligase. In certain embodiments, said method further includes a step of binding of the double stranded DNA nucleic acid, in particular the circular double stranded DNA nucleic acid, to one or more non-nucleic acid moiety (moieties), preferably selected in the group comprising linkers, polypeptides, particles, surfaces, and combinations thereof; so as to generate a functionalized binding the double stranded DNA nucleic acid, in particular a functionalized circular double stranded DNA nucleic acid.

Another aspect of the invention further pertains to a circularized double stranded DNA nucleic acid obtainable by a method according to the instant invention. In some embodiments, said nucleic acid is functionalized and/or relaxed.

A still other aspect of the invention relates to a host cell comprising a circularized double stranded DNA nucleic acid obtainable by a method according to the instant invention.

In a further aspect, the invention pertains to the circularized double stranded DNA nucleic acid according to the instant invention, for use in gene therapy, and/or in DNA vaccination, and/or in cell therapy, and/or in genome editing, and/or in the production of induced pluripotent stem cells, and/or in the transfection or transformation of cultured cells.

One aspect of the invention relates to the use of the circularized double stranded DNA nucleic acid according to the instant invention, for the storage of data, and/or for the sequencing of nucleic acids, and/or for the production of rolling circle DNA, and/or for the production of proteins, and/or for the production of RNA, and/or in metabolic pathway engineering, and/or in molecular biology, and/or for the transformation of bacteria, and/or for the production of viruses.

The invention also relates to a kit for the circularization of a double stranded DNA nucleic acid, said kit comprising:
a) a polypeptide having a 3′-5′ nuclease activity;
b) a polypeptide having a 5′-3′ nuclease activity;
c) a DNA polymerase;
d) a mesophilic DNA ligase; and optionally,
e) one or more buffer(s).

In some embodiments, said kit comprises at least two vials:
the first vial comprising the polypeptide having a 3′-5′ nuclease activity; and optionally alkaline phosphatase and/or a polypeptide with a type I exonuclease activity;
the second vial comprising a DNA ligase; and optionally a polypeptide having a 5′-3′ nuclease activity, and/or a DNA polymerase with no strand displacement activity and with no 5′ to 3′ exonuclease activity, ATP and dNTPs.

In certain embodiments, said kit further comprises one or more primer(s) selected in the group consisting of a primer of sequence SEQ ID NO. 43 and a primer of sequence SEQ ID NO. 44.

### Definitions

In the present invention, the following terms have the following meanings:
“About” preceding a number encompasses plus or minus 10%, or less, of the value of said number. It is to be understood that the value to which the term “about” refers to is itself also specifically, and preferably, disclosed.
“In vitro” when referred to a method, is intended to mean that the steps of the method are performed without the requirement of a living organism, such as, e.g., a bacterium. In some embodiments, the method may be “substantially performed in vitro”, meaning that most of the steps do not require the presence of a living organism, such as, e.g., a bacterium, and that at least one step of the method may optionally be performed in the presence of said living organism.
“Comprises” is intended to mean “contains”, “encompasses” and “includes”. In some embodiments, the term “comprises” also encompasses the term “consists of”.
“Amplicon” is intended to relate to a segment of DNA nucleic acid, e.g., chromosomal DNA nucleic acid, that undergoes amplification, in particular by PCR, and contains replicated genetic material. In some embodiments, the term “amplicon” relates to a segment of DNA nucleic acid that derives from two or more segments, which can be single stranded or double stranded DNA molecules, intentionally assembled together during the PCR reaction.
“Double stranded nucleic acid” is intended to refer to a nucleic acid wherein the two strands, in the 5′-3′ and 3′-5′ orientations, are complementary to one another and hybridize with one another. As used herein, the term “double stranded nucleic acid” further refers to a “double stranded nucleic acid molecule”, as a biological entity.
“Intramolecularly annealed dsDNA nucleic acid” or “product of intramolecular annealing of DNA nucleic acid” refers to the DNA nucleic acid obtained after joining two ends of the same DNA molecule through the hydrogen bonds formed by annealing of the terminal single stranded overlapping overhangs. This “circular” structure of DNA nucleic acids contains 2 gaps, or 2 nicks, or 1 gap and 1 nick, wherein both strains are discontinuous. As used herein, the term “intramolecularly annealed dsDNA nucleic acid” further refers to a “intramolecularly annealed dsDNA nucleic acid molecule”, as a biological entity.
“Circularized double stranded DNA nucleic acid” refers to a dsDNA nucleic acid that contains one or two continuous strands. As used herein, the term “circularized double stranded DNA nucleic acid” further refers to a “circularized double stranded DNA nucleic acid molecule”, as a biological entity.
“Continuous strand” refers to a strand of a nucleic acid that contains no gap and/or no nick. In other words, the nucleotides are linked one to another by a 5′-3′ phosphate bond. In other words, each of the nucleotide of the continuous strand is linked to at least one, in particular two, other nucleotide(s).
“Discontinuous strand” refers to a strand of a nucleic acid that contains at least one gap and/or at least one nick.
“Hybridization” is intended to refer to the action for two complementary nucleic acid strands, i.e. the strand in the 5′-3′ orientation and the complementary strand in the 3′-5′ orientation, to contact in a sense/antisense manner and to form hydrogen bonds.
“Homologous regions” is intended to refer to regions displaying similarity of nucleotide sequences. Within the scope of the invention the term “similarity” also encompasses the term “identity”. In practice, homologous regions may hybridize with one another in a sense/antisense manner Oppositely, “Non-homologous regions” is intended to refer to regions that do not share any sequence similarity or identity. In practice, non-homologous regions cannot hybridize with one another in a sense/antisense manner. The level of identity/similarity of two nucleic acid sequences may be performed by using any one of the known algorithms available from the state of the art. Illustratively, the nucleic acid identity percentage may be determined using the CLUSTAL W software (version 1.83) the parameters being set as follows:
   for slow/accurate alignments: (1) Gap Open Penalty: 15; (2) Gap Extension Penalty: 6.66; (3) Weight matrix: IUB;
   for fast/approximate alignments: (4) K-tuple (word) size: 2; (5) Gap Penalty: 5; (6) No. of top diagonals: 5; (7) Window size: 4; (8) Scoring Method: PERCENT.
“Identical regions” is intended to refer to regions within a nucleic acid sharing the same nucleotide sequence. Oppositely, “Distinct” is intended to refer to regions within a nucleic acid that do not have identical nucleotide sequences.
“Recessing” is intended to refer to the action of removing, e.g. by enzymatic digestion, one strand of the extremity of a double stranded nucleic acid.
“Overlapping overhangs” is intended to refer to single stranded extremities of nucleic acids that may hybridize with one another.
“Annealing” or “anneal” is intended to refer to the action of hybridizing 2 complementary nucleic acid strands in a sense/antisense manner
“Site of restriction” is intended to refer to a nucleic acid sequence capable to bind to the corresponding restriction enzyme and to be cleaved in suitable conditions. As used herein, a site of restriction includes the recognition/cleaving site as well as the additional surrounding nucleotide(s) that are required for the enzyme to bind and cleave.
“Nick” is intended to refer to a discontinuity in a double stranded DNA nucleic acid where there is no phosphodiester bond between adjacent nucleotides of one strand. Within the scope of the instant invention, the terms “nick” and “break” are equivalent and may substitute one another.
“Nickase”, “nicking enzyme” and “nicking (endo)nuclease” are meant to substitute one another and are intended to refer to a polypeptide capable of generating a nick.
“Gap” is intended to refer to a single-stranded region within a double-stranded DNA nucleic acid delimited by two double-stranded regions.
“Circularized” is intended to refer to the structure of the final product obtainable by the method according to the invention. In other words, a circularized double-stranded DNA nucleic acid according to the invention refers to a dsDNA nucleic acid that has been circularized following by the method according to the invention.
“Functionalized nucleic acid” is intended to refer to a nucleic acid that is bound or conjugated to one or more non-nucleic acid moiety (moieties), in particular one or more functional polypeptide(s). As used herein the functional moiety or polypeptide may facilitate the penetration of nucleic acid molecules into a target recipient cell, facilitate the nuclear localization of the nucleic acid molecules into a target recipient cell, facilitate the localization in cell organelles of the nucleic acid molecules into a target recipient cell, facilitate the visualization of the nucleic acid and/or facilitate the binding to epitopes of choice on cell surface. As used herein, the term “bound” refers to covalent or non-covalent attachment of the moiety or polypeptide to the nucleic acid according to the invention. As used herein, the term “conjugated” is intended to mean that the nucleic acid and the functional moiety or polypeptide are attached one to another by the means of a covalent bond. As used herein, the term “functionalized nucleic acid” further refers to a “functionalized nucleic acid molecule”, as a biological entity.
“Host cell” is intended to refer to a cell that is the recipient of at least one foreign (or exogenous) nucleic acid molecule.
“Treatment”, “treating” or “alleviation” refer to therapeutic treatments wherein the object is to prevent or slow down (lessen) a disorder. Those in need of treatment include those already with the disorder as well as those prone to have the disorder or those in whom the disorder is to be prevented. A subject is successfully “treated” for a disorder if, after receiving a therapeutic amount of a circular double stranded DNA nucleic acid according to the present invention, the subject shows observable and/or measurable reduction in, or absence of, one or more of the following: reduction in the disorder and/or relief to some extent of one or more of the symptoms associated with the disorder; reduced morbidity and mortality, and/or improvement in quality of life issues. The above parameters for assessing successful treatment and improvement in the disorder are readily measurable by routine procedures familiar to a physician.
“Prevention” refers to preventing or avoiding the occurrence of one or more symptom(s) of a disorder. In the present invention, the term “prevention” may refer to a secondary prevention, i.e. to the prevention of the re-occurrence of a symptom or a relapse of a disorder. It may also refer, when the disease is cancer, to the prevention of occurrence of metastases after the treatment and/or the removal of a tumor.
“Therapeutic effective amount” refers to an amount sufficient to effect beneficial or desired results including clinical results. A therapeutic effective amount can be administered in one or more administrations.
“Individual” refers to a vertebrate animal, preferably a mammal, more preferably a human. Examples of individuals include humans, non-human primates, dogs, cats, mice, rats, horses, cows, sheep and transgenic species thereof. In one embodiment, an individual may be a “patient”, i.e. a warm-blooded animal, more preferably a human, who/which is awaiting the receipt of, or is receiving medical care or was/is/will be the object of a medical procedure, or is monitored for the development of a disease. In one embodiment, the individual is an adult (for example a human subject above the age of 18). In another embodiment, the individual is a child (for example a human subject below the age of 18). In one embodiment, the individual is a male. In another embodiment, the individual is a female.

### DETAILED DESCRIPTION

The inventor showed that it is possible to easily and rapidly circularize double stranded nucleic acids, starting from any double stranded nucleic acid molecule, in particular, any linear or linearized circular double stranded nucleic acid molecule. The method of the invention may be performed within a couple of hours through self-hybridization of DNA monomers, and provides one to five orders of magnitude higher yields with up to about 90% efficiency within the range of 100 ng to 10 μg or more, as compared to other methods known in the art. It is worth mentioning that methods known in the art for obtaining circular DNA molecules through hybridization of two or more different DNA monomers, i.e. through DNA assembly, merely achieve from about 0.01% to about 10% efficiency within the range of 10 pg to 10 ng of circular DNA molecules, and in most cases require an additional step of amplification of those generated circular DNA nucleic acids by the means of a bacterial system. The present invention provides, for the first time, a method for the production, in particular for the in vitro production, of circular DNA nucleic acids, analogous to DNA minicircles obtained by bacterial systems. The invention also provides circular DNA nucleic acids that may be easily functionalized by conjugation to peptides or proteins, such as, for example, antibodies, and the like. To date, functionalization of circular DNA nucleic acids is often required to be performed in living organisms.

Finally, the circularized double stranded DNA nucleic acids obtained by the method of the invention may find many applications, as such as, e.g., cell transfection, gene therapy, storage of information in DNA molecules and for any other use wherein fast and efficient DNA circularization is needed.

In one aspect, the invention relates to a method for the circularization of a double stranded DNA nucleic acid, said method comprising the steps of:
a) providing a linear or circular double stranded DNA nucleic acid comprising a nucleic acid of the following formula (I):

NHR1-SOR1ₖ-HR1-TBC-HR2-SOR2_{y}-NHR2 (I),

wherein:
TBC represents a core double stranded DNA nucleic acid to be circularized, in particular having a length of at least about 250 bp, preferably at least about 500 bp, more preferably at least about 1,000 bp;
HR1 and HR2 represent identical homologous double stranded DNA nucleic acids of identical orientation;
SOR1ₓ and SOR2_{y} represent nucleic acids having a length of about 5 bp to about 60 bp comprising a site of restriction capable of generating 3′ overhangs having a length of at least 4 nucleotides and having as 3′ terminal nucleotide A, T or G; and x and y are 0 or 1; when x and y are 1, TBC further comprises 2 sites of nicking restriction each located about 0 nucleotide to about 100 nucleotides respectively from the 3′ end of HR1 and from the 3′ end of HR2;
NHR1 and NHR2 represent distinct non-homologous double stranded DNA nucleic acids having a length of 0 bp to about 200 bp;
b1) when x and y are 0, digesting the circular double stranded DNA nucleic acid comprising a nucleic acid of formula (I) from step a), in the presence of a restriction enzyme that cleaves a restriction site that is not located in any one of HR1, HR2 and TBC, so as to obtain a linearized nucleic acid;
b2) when x and y are 1,
   optionally digesting the circular double stranded DNA nucleic acid comprising a nucleic acid of formula (I) from step a), in the presence of a restriction enzyme that cleaves a restriction site that is not located in any one of HR1, HR2 and TBC, so that to generate a linearized nucleic acid;
   digesting the circular, linear or linearized nucleic acid in the presence of (i) a polypeptide with nickase activity capable of introducing a nick within TBC, and (ii) restriction enzyme(s) capable of digesting the corresponding site of restriction in SOR1 and SOR2;
c) recessing both ends of identical orientation of the nucleic acid obtained at step b1) or step b2), in the presence of a polypeptide having a 3′-5′ nuclease activity, so that HR1 and HR2 are capable of forming overlapping overhangs;
d) annealing the DNA nucleic acid obtained at step c), thereby generating 2 gaps, 2 nicks, or 1 nick and 1 gap;
e) filling the 1 or 2 gaps generated at step d), step e) being optional when 2 nicks are generated at step d);
f) optionally removing the NHR1 and NHR2 at both 5′ ends, when the length of the NHR1 and NHR2 is superior to 0 bp;
g) sealing at least one nick, so as to obtain a circularized double stranded DNA nucleic acid, in which at least one strand is continuous.

In some embodiments, the method is an in vitro method. In certain embodiments, the method is a substantially in vitro method. In some embodiments, the method is performed substantially in vitro. In certain embodiments, the method is performed exclusively in vitro.

In certain embodiments, the linear or circular double stranded DNA nucleic acid has a length of at least about 250 bp, preferably at least about 500 bp, more preferably at least about 1,000 bp.

Within the scope of the instant invention, the expression “at least about 250 bp” encompasses 250, 300, 350, 400, 450, 500, 550, 600, 650, 700, 750, 800, 850, 900, 950, 1,000, 1,250, 1,500, 1,750, 2,000, 2,250, 2,500, 2,750, 3,000, 3,500, 4,000, 4,500, 5,000, 5,500, 6,000, 6,500, 7,000, 7,500, 8,000, 8,500, 9,000, 9,500, 10,000, 12,000, 14,000, 16,000, 18,000, 20,000, 25,000, 30,000, 35,000, 40,000, 45,000, 50,000 bp, or more.

In some embodiments, SOR1 and SOR2 represent a nucleic acid having a length of about 5 bp to about 60 bp, preferably of about 7 bp to about 60 bp.

Within the scope of the instant invention, the term “about 5 bp to about 60 bp” includes 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 and 60 bp.

As used herein, the “sites of restriction capable of generating 3′ overhangs having a length of at least 4 nucleotides and having as 3′ terminal nucleotide A, T or G” comprised in SOR1 and SOR2 include the recognition/cleaving sites as well as the additional surrounding nucleotide(s) that are required for the enzyme to bind and to efficiently cleave. In some embodiments, the recognition/cleaving site is surrounded by 1, 2, 3, 4, 5, 6 or more extra nucleotides at its 5′ end and/or its 3′ end, which extra nucleotide(s) participate(s) to the binding of the enzyme of restriction and to the efficient cleaving.

In certain embodiments, x and y have the same value, which encompasses 0 and 1. In other words, x and y are 0 and alternatively, x and y are 1.

In some embodiments, TBC comprises 2 sites of nicking restriction each located about 0 nucleotide to about 100 nucleotides respectively from the 3′ end of HR1 and from the 3′ end of HR2.

Within the scope of the instant invention, the term “0 nucleotide to about 100 nucleotides” includes 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and 100 nucleotides.

In some embodiments, the locations of the 2 sites of nicking restriction are independent with respect to one another. Illustratively, the positions of the 2 sites of nicking restriction with respect to the 3′ end of HR1 and the 3′ end of HR2 may be identical or alternatively distinct.

In certain embodiments, NHR1 and NHR2 represent non-homologous double stranded DNA nucleic acids having a length of 0 bp to about 200 bp.

Within the scope of the instant invention, the term “0 bp to about 200 bp” includes 0, 1, 2, 3, 4, 5, 6, 7, 8, 9, 10, 11, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 110, 115, 120, 125, 130, 135, 140, 145, 150, 155, 160, 165, 170, 175, 180, 185, 190, 195 and 200 bp.

In some embodiments, at least TBC, HR1, HR2 from the linear or circular double stranded nucleic acid of formula (I) are from eukaryotic origin. In some embodiments, TBC, HR1 and/or HR2 from the linear or circular double stranded nucleic acid of formula (I) are from microbial origin. In said embodiments, TBC, HR1 and/or HR2 may comprise one or more origin(s) of replication from bacterial origin (e.g. origins of replication pMB1, ColE1 or f1) and/or from viral origin (e.g., SV40 origin of replication functional in HEK293T cell line). In said embodiments, TBC, HR1 and/or HR2 may further comprise one or more nucleic acid(s) encoding one or more polypeptide(s) involved in the resistance to one or more antibiotic(s).

In certain embodiments the TBC, HR1 and/or HR2 may comprise a S/MAR (for scaffold/matrix attachment region) nucleic acid sequence. As used herein, the S/MAR nucleic acid sequence, also referred to SAR (for scaffold-attachment region) or MAR (for matrix-associated region), are intended to refer to a nucleic acid sequence that is naturally found in the DNA of eukaryotic chromosomes and that promotes attachment to the nuclear matrix. In some embodiments, the S/MAR nucleic acid sequence may serve as an origin of replication. In practice, circularized nucleic acid molecules comprising a S/MAR nucleic acid sequence may behave as extra-chromosome in a transfected cell and be advantageously transmitted to its progeny. Illustratively, suitable S/MAR nucleic acid sequence may be determined as described in Narwade et al. (Nucleic Acids Research, 2019; 47(14):7247-7261).

In practice, the S/MAR nucleic acid sequence has a length comprised from about 50 bp to about 2,000 bp, preferably from about 400 bp to about 1,500 bp. Within the scope of the invention, the expression “about 50 bp to about 2,000 bp” 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 300, 400, 500, 600, 700, 800, 900, 1,000, 1,100, 1,200, 1,300, 1,400, 1,500, 1,600, 1,700, 1,800, 1,900 and 2,000 bp.

It is to be understood that upon circularization, the circularized nucleic acids according to the instant invention may be compatible with a replication step in a eukaryotic cell, and/or in a bacterial cell. In some embodiments, the circularized nucleic acids according to the instant invention may be compatible with a replication step in cell-free system, such as, e.g., the OriCiro® technology (see https://www.oriciro.com/).

In certain embodiments, TBC is a nucleic acid encoding a polypeptide of therapeutic and/or economic interest. Non-limitative examples of polypeptide of therapeutic and/or economic interest include antibiotics, anti-fungal compounds, anti-viral compounds, fluorescent polypeptides, growth factors, antibodies (e.g., therapeutic and/or diagnosis antibodies), hormones, cytokines, enzymes, plasmatic factors (e.g., clotting factors), metabolic pathways factors, and the like.

In certain embodiments, step a) is performed with an amount of linear or circular double stranded DNA nucleic acid comprising a nucleic acid of formula (I) comprised from about 0.1 ng to about 100 mg. Within the scope of the instant invention, the term “about 0.1 ng to about 100 μg” encompasses 0.1 ng, 0.5 ng, 1 ng, 5 ng, 10 ng, 15 ng, 20 ng, 25 ng, 30 ng, 35 ng, 40 ng, 45 ng, 50 ng, 55 ng, 60 ng, 65 ng, 70 ng, 75 ng, 80 ng, 85 ng, 90 ng, 95 ng, 100 ng, 150 ng, 200 ng, 250 ng, 300 ng, 350 ng, 400 ng, 450 ng, 500 ng, 550 ng, 600 ng, 650 ng, 700 ng, 750 ng, 800 ng, 850 ng, 900 ng, 950 ng, 1 μg, 2 μg, 3 μg, 4 μg, 5 μg, 6 μg, 7 μg, 8 μg, 9 μg, 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 200 μg, 300 μg, 400 μg, 500 μg, 600 μg, 700 μg, 800 μg, 900 μg, 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg and 100 mg. In some embodiments, step a) is performed with an amount of linear or circular double stranded DNA nucleic acid comprising a nucleic acid of formula (I) comprised from about 500 ng to about 15 μg.

In some embodiments, the linear double stranded DNA nucleic acid of step a) is an amplicon obtained by polymerase chain reaction (PCR).

In some embodiments, the NHR1 and NHR2 represent non-homologous double stranded DNA nucleic acids having a length of 1 bp to about 200 bp. Illustratively, a DNA nucleic acid of formula (1a)-(1o) may be amplified with selected primers in order to achieve the linear double stranded DNA nucleic acid of step a) (see Table 1 below).

TABLE 1

| Starting DNA nucleic acid | Primers for PCR amplification |
|---|---|
| TBC (1a) | 5′-3′ primer(s) comprise(s) NHR1-SOR1ₓ-HR1 |
| | 3′-5′ primer(s) comprise(s) HR2-SOR2_{y}-NHR2 |
| HR1-TBC (1b) | 5′-3′ primer(s) comprise(s) NHR1-SOR1ₓ |
| | 3′-5′ primer(s) comprise(s) HR2-SOR2_{y}-NHR2 |
| SOR1ₓ-HR1-TBC (1c) | 5′-3′ primer(s) comprise(s) NHR1 |
| | 3′-5′ primer(s) comprise(s) HR2-SOR2_{y}-NHR2 |
| NHR1-SOR1ₓ-HR1-TBC (1d) | 3′-5′ primer(s) comprise(s) HR2-SOR2_{y}-NHR2 |
| TBC-HR2 (1e) | 5′-3′ primer(s) comprise(s) NHR1-SOR1ₓ-HR1 |
| | 3′-5′ primer(s) comprise(s) SOR2_{y}-NHR2 |
| TBC-HR2-SOR2_{y} (1f) | 5′-3′ primer(s) comprise(s) NHR1-SOR1x-HR1 |
| | 3′-5′ primer(s) comprise(s) NHR2 |
| TBC-HR2-SOR2_{y}-NHR2 (1g) | 5′-3′ primer(s) comprise(s) NHR1-SOR1ₓ-HR1 |
| HR1-TBC-HR2 (1h) | 5′-3′ primer(s) comprise(s) NHR1-SOR1ₓ |
| | 3′-5′ primer(s) comprise(s) NHR2-SOR2_{y} |
| SOR1ₓ-HR1-TBC-HR2 (1i) | 5′-3′ primer(s) comprise(s) NHR1 |
| | 3′-5′ primer(s) comprise(s) NHR2-SOR2_{y} |
| HR1-TBC-HR2-SOR2_{y} (1j) | 5′-3′ primer(s) comprise(s) NHR1-SOR1ₓ |
| | 3′-5′ primer(s) comprise(s) NHR2 |
| SOR1ₓ-HR1-TBC-HR2-SOR2_{y} | 5′-3′ primer(s) comprise(s) NHR1 |
| (1k) | 3′-5′ primer(s) comprise(s) NHR2 |
| NHR1-SOR1ₓ-HR1-TBC-HR2 (1l) | 3′-5′ primer(s) comprise(s) SOR2_{y}-NHR2 |
| NHR1-SOR1ₓ-HR1-TBC-HR2- | 3′-5′ primer(s) comprise(s) NHR2 |
| SOR2_{y} (1m) | |
| HR1-TBC-HR2-SOR2_{y}-NHR2 (1n) | 5′-3′ primer(s) comprise(s) NHR1-SOR1ₓ |
| SOR1ₓ-HR1-TBC-HR2-SOR2_{y}- | 5′-3′ primer(s) comprise(s) NHR1 |
| NHR2 (1o) | |

It is understood that the primers suitable to implement the invention also comprise at least 5 nucleotides that hybridize to the starting DNA nucleic acid, as disclosed herein.

In some embodiments, when the NHR1 and NHR2 have a length of 0 bp, and when SOR1 and SOR2 are absent (x and y are 0), the linear double stranded DNA nucleic acid of step a) is a linear double stranded DNA nucleic acid comprising a nucleic acid of formula (2):

HR1-TBC-HR2 (2).

Illustratively, a DNA nucleic acid of formula (2a), (2b) or (2c) may be amplified with selected primers in order to achieve the linear double stranded DNA nucleic acid of step a) (see Table 2 below).

TABLE 2

| Starting DNA nucleic acid | Primers for PCR amplification |
|---|---|
| TBC (2a) | 5′-3′ primer(s) comprise(s) HR1 |
| | 3′-5′ primer(s) comprise(s) HR2 |
| HR1-TBC (2b) | 3′-5′ primer(s) comprise(s) HR2 |
| TBC-HR2 (2c) | 5′-3′ primer(s) comprise(s) HR1 |

In some embodiments, when the NHR1 and NHR2 have a length of 0 bp, the linear double stranded DNA nucleic acid of step a) is a linear double stranded DNA nucleic acid comprising a nucleic acid of formula (3):

SOR1ₓ-HR1-TBC-HR2-SOR2_{y} (3).

Illustratively, a DNA nucleic acid of formula (3a)-(3 h) may be amplified with selected primers in order to achieve the linear double stranded DNA nucleic acid of step a) (see Table 3 below).

TABLE 3

| Starting DNA nucleic acid | Primers for PCR amplification |
|---|---|
| TBC (3a) | 5′-3′ primer(s) comprise(s) SOR1ₓ-HR1 |
| | 3′-5′ primer(s) comprise(s) HR2-SOR2_{y} |
| HR1-TBC (3b) | 5′-3′ primer(s) comprise(s) SOR1ₓ |
| | 3′-5′ primer(s) comprise(s) HR2-SOR2_{y} |
| SOR1ₓ-HR1-TBC (3c) | 3′-5′ primer(s) comprise(s) HR2-SOR2_{y} |
| TBC-HR2 (3d) | 5′-3′ primer(s) comprise(s) SOR1ₓ-HR1 |
| | 3′-5′ primer(s) comprise (s) SOR2_{y} |
| TBC-HR2-SOR2_{y} (3e) | 5′-3′ primer(s) comprise(s) SOR1ₓ-HR1 |
| HR1-TBC-HR2 (3f) | 5′-3′ primer(s) comprise(s) SOR1ₓ |
| | 3′-5′ primer(s) comprise(s) SOR2_{y} |
| SOR1ₓ-HR1-TBC-HR2 (3g) | 3′-5′ primer(s) comprise(s) SOR2_{y} |
| HR1-TBC-HR2-SOR2_{y} (3h) | 5′-3′ primer(s) comprise(s) SOR1ₓ |

In some embodiments, when NHR1 has a length of 0 bp, and NHR2 represents non-homologous double stranded DNA nucleic acid having a length of 1 bp to about 200 bp, the linear double stranded DNA nucleic acid of step a) is a linear double stranded DNA nucleic acid comprising a nucleic acid of formula (4):

SOR1ₓ-HR1-TBC-HR2-SOR2_{y}-NHR2 (4).

Illustratively, a DNA nucleic acid of formula (4a)-(4j) may be amplified with selected primers in order to achieve the linear double stranded DNA nucleic acid of step a) (see Table 4 below).

TABLE 4

| Starting DNA nucleic acid | Primers for PCR amplification |
|---|---|
| TBC (4a) | 5′-3′ primer(s) comprise(s) SOR1ₓ-HR1 |
| | 3′-5′ primer(s) comprise(s) HR2- SOR2_{y}-NHR2 |
| HR1-TBC (4b) | 5′-3′ primer(s) comprise(s) SOR1ₓ |
| | 3′-5′ primer(s) comprise(s) HR2- SOR2_{y}-NHR2 |
| SOR1ₓ-HR1-TBC (4c) | 3′-5′ primer(s) comprise(s) HR2- SOR2_{y}-NHR2 |
| TBC-HR2 (4d) | 5′-3′ primer(s) comprise(s) SOR1ₓ-HR1 |
| | 3′-5′ primer(s) comprise(s) SOR2y-NHR2 |
| TBC-HR2-SOR2_{y} (4e) | 5′-3′ primer(s) comprise(s) SOR1ₓ-HR1 |
| | 3′-5′ primer(s) comprise(s) NHR2 |
| TBC-HR2-SOR2_{y}-NHR2 (4f) | 5′-3′ primer(s) comprise(s) SOR1ₓ-HR1 |
| HR1-TBC-HR2-SOR2_{y}-NHR2 (4g) | 5′-3′ primer(s) comprise(s) SOR1ₓ |
| HR1-TBC-HR2 (4h) | 5′-3′ primer(s) comprise(s) SOR1ₓ |
| | 3′-5′ primer(s) comprise(s) SOR2_{y}-NHR2 |
| SOR1ₓ-HR1-TBC-HR2 (4i) | 3′-5′ primer(s) comprise(s) SOR2_{y}-NHR2 |
| SOR1ₓ-HR1-TBC-HR2-SOR2_{y} (4j) | 3′-5′ primer(s) comprise(s) NHR2 |

In some embodiments, when NHR1 represents a non-homologous double stranded DNA nucleic acid having a length of 1 bp to about 200 bp, and NHR2 has a length of 0 bp, the linear double stranded DNA nucleic acid of step a) is a linear double stranded DNA nucleic acid comprising a nucleic acid of formula (5):

NHR1-SOR1ₓHR1-TBC-HR2-SOR2_{y}(5).

Illustratively, a DNA nucleic acid of formula (5a)-(5j) may be amplified with selected primers is order to achieve the linear double stranded DNA nucleic acid of step a) (see Table 5 below).

TABLE 5

| Starting DNA nucleic acid | Primers for PCR amplification |
|---|---|
| TBC (5a) | 5′-3′ primer(s) comprise(s) NHR1-SOR1ₓ-HR1 |
| | 3′-5′ primer(s) comprise(s) HR2-SOR2_{y} |
| HR1-TBC (5b) | 5′-3′ primer(s) comprise(s) NHR1-SOR1ₓ |
| | 3′-5′ primer(s) comprise(s) HR2-SOR2_{y} |
| SOR1ₓ-HR1-TBC (5c) | 5′-3′ primer(s) comprise(s) NHR1 |
| | 3′-5′ primer(s) comprise(s) HR2-SOR2_{y} |
| NHR1-SOR1ₓ-HR1-TBC (5d) | 3′-5′ primer(s) comprise(s) HR2-SOR2_{y} |
| TBC-HR2 (5e) | 5′-3′ primer(s) comprise(s) NHR1- SOR1ₓ-HR1 |
| | 3′-5′ primer(s) comprise(s) SOR2_{y} |
| TBC-HR2-SOR2_{y} (5f) | 5′-3′ primer(s) comprise(s) NHR1- SOR1ₓ-HR1 |
| HR1-TBC-HR2 (5g) | 5′-3′ primer(s) comprise(s) NHR1-SOR1ₓ |
| | 3′-5′ primer(s) comprise(s) SOR2_{y} |
| SOR1ₓ-HR1-TBC-HR2 (5h) | 5′-3′ primer(s) comprise(s) NHR1 |
| | 3′-5′ primer(s) comprise(s) SOR2_{y} |
| NHR1-SOR1ₓ-HR1-TBC-HR2 | 3′-5′ primer(s) comprise(s) SOR2_{y} |
| (5i) | |
| SOR1ₓ-HR1-TBC-HR2-SOR2_{y} | 5′-3′ primer(s) comprise(s) NHR1 |
| (5j) | |

In some embodiments, the amplicons are raw, i.e., non-purified. In some alternative embodiments, the amplicons are purified. Methods for purifying amplicons are known in the state of the art (see, e.g., Sambrook et al. (Molecular Cloning—A Laboratory Manual—2^{nd} Edition; Cold Spring Harbor Laboratory Press)). Purification may include phenol/chloroform, and/or ethanol precipitation. Purification may also be performed with an appropriate commercial kit, such as, e.g., Monarch® PCR & DNA Cleanup kit from NEB®, StrataPrep® PCR purification kit from Agilent®, following the manufacturer's instructions.

In certain embodiments, the linear double stranded DNA nucleic acid of step a) may be obtained by amplification and by digestion by the mean of one or more restriction enzymes.

In certain embodiments, the circular double stranded DNA nucleic acid comprising a nucleic acid of formula (I) is selected in the group consisting of a chromosome, a plasmid, a cosmid, a bacterial artificial chromosome (BAC), and the like.

As used herein, the term “chromosome” refers to a genomic DNA nucleic acid molecule, originating from a micro-organism or a eukaryote.

As used herein, the term “plasmid” refers to a small extra-genomic DNA nucleic acid molecule, most commonly found as circular double stranded DNA nucleic acid molecules that may for example be used as a cloning vector in molecular biology, to make and/or modify copies of DNA fragments up to about 15 kb (i.e., 15,000 base pairs). Plasmids may also be used as expression vectors to produce large amounts of proteins of interest encoded by a nucleic acid sequence found in the plasmid downstream of a promoter sequence.

As used herein, the term “cosmid” refers to a hybrid plasmid that contains cos sequences from Lambda phage, allowing packaging of the cosmid into a phage head and subsequent infection of bacterial cell wherein the cosmid is cyclized and can replicate as a plasmid. Cosmids are typically used as cloning vector for DNA fragments ranging in size from about 32 kb to about 52 kb.

As used herein, “bacterial artificial chromosome” or “BAC” refers to an extra-genomic nucleic acid molecule based on a functional fertility plasmid that allows the even partition of said extra-genomic DNA molecules after division of the bacterial cell. BACs are typically used as cloning vector for DNA fragment ranging in size from about 150 kb to about 350 kb.

In some embodiments, the nucleic acid of formula (I) may be a synthetic nucleic acid, i.e., obtained de novo by chemical synthesis, by any suitable method known from the state of the art, or a method adapted therefrom.

In practice, because NHR1 and NHR2 are distinct non-homologous nucleic acids, they cannot hybridize with each other, with HR1, with HR2 and with any part of TBC in suitable conditions. Inversely, because HR1 and HR2 represent identical homologous double stranded DNA nucleic acids of identical orientation, they may hybridize with each other in suitable condition.

In some embodiments, HR1 and HR2 display a ΔG ranging from about 20 to about 60. Within the scope of the instant invention, the term “from about 20 to about 60” include 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 and 60. In practice, measuring the ΔG parameter may be performed by any suitable method known from the state of the art, or a method adapted therefrom. Illustratively, measuring the ΔG parameter may be performed as disclosed in Wang et al. (Nat Commun 7, 10319 (2016)).

In some embodiments, the linear double stranded DNA nucleic acid of step a) further comprises a 5′-phosphate at each 5′ ends.

In some embodiments, NHR1 and/or HR1, on one hand, and NHR2 and/or HR2, on the other hand, further comprise one or more thiophosphate nucleotide(s). As used herein, the term “thiophosphate nucleotide” refers to a nucleotide analogue possessing a thio-phosphate bond (PO₃S) in lieu of a phosphate bond (PO₄). In practice, thiophosphate nucleotides may be commercially available, e.g., from Sigma Aldrich®.

In certain embodiments, the linear double stranded DNA nucleic acid of step a) comprises a 5′-phosphate at each 5′ ends and one or more thiophosphate nucleotide(s) in NHR1 and/or HR1 and in NHR2 and/or HR2 nucleic acid.

In some embodiments, the 5′-phosphate and/or the thiophosphate nucleotide(s) is/are provided to the linear double stranded DNA nucleic acid by the mean of PCR amplification. In practice, the 5′-phosphate and/or the thiophosphate nucleotide(s) is/are encompassed within the oligonucleotides. In said embodiments, the Pfu DNA polymerase is advantageously used to introduce the 5′-phosphate and/or the thiophosphate nucleotide(s) into the linear double stranded DNA nucleic acid of step a).

In some embodiments, when x and y are 0, digesting the circular double stranded DNA nucleic acid comprising a nucleic acid of formula (I) from step a), is performed in the presence of a restriction enzyme that cleaves a restriction site that is not located in any one of HR1, HR2 and TBC, so as to obtain a linearized nucleic acid.

In said embodiments, the site of restriction may be advantageously located within any one of the NHR1 or NHR2 nucleic acids, or alternatively between the NHR1 and the NHR2 nucleic acids, or anywhere else on the nucleic acid, provided that the restriction site that is not located in any one of nucleic acids HR1, HR2 and TBC.

In certain embodiments, enzymatic digestion may be performed by restriction enzymes capable of generating blunt ends, such as, e.g., AanI, AccII, AcvI, AfeI, AluI, BoxI, BseJI, BspLI, Bst1107I, BsuRI, DpnI, DraI, Ec1136II, EcoRV, Eco147I, EheI, FspAI, HincII, Hpy8I, KspAI, MbiI, MlsI, MssI, NsbI, PdiI, PdmI, Ppu21I, PvuII, RruI, RsaI, RseI, ScaI, SchI, SmaI, SmiI, SnaBI and SspI.

In certain embodiments, enzymatic digestion may be performed also by restriction enzymes capable of generating 5′-hangouts, such as, e.g., Acc65I, AccI, AciI, AcII, AflII, AgeI, AlwI, ApaLI, ApeKI, ApoI, AscI, AseI, AvaI, AvaII, AvrII, BamHI, BanI, BbsI, BbvCI, BbvI, BccI, BceAI, BclI, BcoDI, BfaI, BfuAI, BglII, BlpI, Bpu10I, BsaHI, BsaI, BsaJI, BsaWI, BseYI, BsiWI, BsmAI, BsmBI, BsmFI, BsoBI, BspDI, BspEI, BspHI, BspMI, BspQI, BsrFaI, BsrGI, BssHI, BstBI, BstEII, BstNI, BstYI, Bsu36I, BtgI, BtgZI, ClaI, CviAII, CviQI, DdeI, DpnII, EaeI, EagI, EarI, EcoNI, EcoO109I, EcoRI, Esp3I, FatI, FauI, Fnu4HI, FokI, FspEI, HgaI, HinPlI, HindIII, HinfI, HpaII, Hpy188III, HpyCH4IV, KasI, LpnPI, MboI, MfeI, MluCI, MluI, MseI, MspI, MspJI, NanI, NciI, NcoI, NdeI, NgoMIV, NheI, NotI, PaeR7I, PciII, PflFI, PleI, PpuMI, PspGI, PspOMI, PspXI, RsrII, SalI, SapI, Sau3AI, Sau96I, ScrFI, SexAI, SfaNI, SfcI, SgrAI, SmlI, SpeI, StvD4I, StyI, TaqαI, TfiI, TseI, Tsp45I, TspMI, Tth111I, XbaI, XhoI and XmaI.

In some embodiments, enzymatic digestion may be performed by restriction enzymes resulting in 3′-hangouts that preferentially carry a 3′-terminal C nucleotide, such as, e.g., ApaI, BanII, Bsp1286I, HaeII, KpnI. In some embodiments, the 3′ hangouts are shorter than or equal to 3 nucleotides, i.e. 1, 2 or 3 nucleotides in length.

In some embodiments, enzymatic digestion may be performed by type IIS restriction enzymes such as, e.g., AarI, Acc36I, Ac1WI, AcuI, Alw26I, AlwI, AsuHPI, BbsI, BbvI, BccI, BceAI, BciVI, BcoDI, BfuAI, BfuI, BmrI, BmsI, BmuI, BpiI, BpmI, BpuEI, BsaI, Bse3DI, BseGI, BseMI, BseMII, BseRI. In certain embodiments, enzymatic digestion may be performed by type IIS restriction enzymes such as, e.g., AcuI, AlwI, BaeI, BbsI, BbvI, BccI, BceAI, BcgI, BciVI, BcoDI, BfuAI, BmrI, BpmI, BpuEI, BsaI, BsaXI, BseRI, BsgI, BsmAI, BsmBI, BsmFI, BsmI, BspCNI, BspMI, BspQI, BsrDI, BsrI, BtgZI, BtsCI, BtsI-v2, BtslMutI, CspCI, EarI, EciI, Esp3I, FauI, FokI, HgaI, HphI, HpyAV, MboII, MlyI, MmeI, MnlI, NmeAIII, PleI, SapI and SfaNI.

In some embodiments, when x and y are 1, the method comprises the step(s) of:
optionally digesting the circular double stranded DNA nucleic acid comprising a nucleic acid of formula (I) from step a), in the presence of a restriction enzyme that cleaves a restriction site that is not located in any one of HR1, HR2 and TBC, so that to generate a linearized nucleic acid;
digesting the circular, linear or linearized nucleic acid in the presence of (i) a polypeptide with nickase activity capable of introducing a nick within TBC, and (ii) restriction enzyme(s) capable of digesting the corresponding site of restriction in SOR1 and SOR2.

The optional digestion of the circular double stranded DNA nucleic acid comprising a nucleic acid of formula (I) from step a) may be performed in the presence of any restriction enzyme capable of generating blunt ends or alternatively resulting in 5′-hangouts or alternatively, less preferentially, resulting in 3′-hangouts.

In said embodiments, step b2) results in the generation of a linear double stranded DNA nucleic acid with 3′ overhangs. In practice, the restriction enzyme generates 3′ overhangs having a length of at least 4 nucleotides and having as 3′ terminal nucleotide A, T or G. These 3′ overhangs are not prone to digestion by the polypeptide having a 3′-5′ nuclease activity, in particular Exo III. The polypeptide having a 3′-5′ nuclease activity, in particular Exo III, would recess the 3′ ends from the nick location.

In some embodiments, the polypeptide with nickase activity is selected in the group consisting of Nt.BspQI, Nt.CviPII, Nt.BstNBI, Nb.BsrDI, Nb.BtsI, Nt.AlwI, Nb.BbvCI, Nt.BbvCI, Nb.BsmI, Nb.BssSI and Nt.BsmAI. In practice, the polypeptide with nickase activity may be purchased from NEB®.

In practice, about 1 μg to about 5 μg of DNA nucleic acids may be treated with about 0.1u to about 5u of a polypeptide with nickase activity. In practice, treatment with a polypeptide with a nickase activity is or may be performed for about 30 min to about 180 min, preferably for about 60 min to about 90 min. In practice, treatment with a polypeptide with a nickase activity is or may be performed at a temperature of about 0° C. to about 70° C., preferably at a temperature of about 35° C. to about 55° C. A suitable buffer may be composed of 100 min NaCl, 50 min Tris-HCl pH 7.9 at 25° C., 10 min MgCl₂, 100 μg/ml BSA (provided by NEB as Buffer 1×NEBuffer® 3.1) or composed of 50 mM Potassium Acetate, 20 mM Tris-acetate pH 7.9 at 25° C., 10 mM Magnesium Acetate, 100 μg/ml BSA (provided by NEB as Buffer 1× CutSmart® Buffer) or composed for the optimal efficiency of nickase activity.

In practice, 1u (1 unit) of Nt.BspQI may be defined as the amount of enzyme required to convert 1 μg of supercoiled pUC19 DNA to open circular form in 1 hour at 50° C. in a total reaction volume of 50 μl. 1u (1 unit) of Nt.CviPII may be defined as the amount of enzyme required to digest 1 μg of pUC19 DNA resulting in a stable pattern of fragments between 25 and 200 bp in 1 hour at 37° C. in a total reaction volume of 50 μl. 1u (1 unit) of Nt.BstNBI may be defined as the amount of enzyme required to digest 1 μg T7 DNA in 1 hour at 55° C. in a total reaction volume of 50 μl. 1u (1 unit) of Nb.BsrDI may be defined as the amount of enzyme required to convert 1 μg of supercoiled pUC19 DNA to open circular form in 1 hour at 65° C. in a total reaction volume of 50 μl. 1u (1 unit) of Nb.BtsI may be defined as the amount of enzyme required to convert 1 μg of supercoiled plasmid ΦX174 RF I DNA to open circular form in 1 hour at 37° C. in a total reaction volume of 50 μl. 1u (1 unit) of Nt.AlwI may be defined as the amount of enzyme required to convert 1 μg of supercoiled pUC101 DNA (dam−/dcm−) to open circular form in 1 hour at 37° C. in a total reaction volume of 50 μl. 1u (1 unit) of Nb.BbvCI or Nt.BbvCI may be defined as the amount of enzyme required to convert 1 μg of supercoiled plasmid DNA to open circular form in 1 hour at 37° C. in a total reaction volume of 50 μl. 1u (1 unit) of Nb.BsmI may be defined as the amount of enzyme required to convert 1 μg of supercoiled plasmid pBR322 DNA to open circular form in 1 hour at 65° C. in a total reaction volume of 50 μl. 1u (1 unit) of Nb.BssSI may be defined as the amount of enzyme required to convert 1 μg of supercoiled pUC19 DNA to open circular form in 1 hour at 37° C. in a total reaction volume of 50 μl. 1u (1 unit) of Nt.BsmAI may be defined as the amount of enzyme required to convert 1 μg of supercoiled plasmid DNA to open circular form in 1 hour at 37° C. in a total reaction volume of 50 μl.

In certain embodiments, the restriction enzyme capable of generating 3′ overhangs having a length of at least 4 nucleotides and having as 3′ terminal nucleotide A, T or G is selected in the group consisting of AatII, AjuI, AloI, ArsI, Bael, BarI, BmtI, BplI, BspMAI, BspOI, BstNSI, BstXI, EcoT22I, FaeI, FalI, FseI, GsaI, HgiAI, HinlII, Hpy99I, Hsp92II, I-CeuI, I-PpoI, I-SceI, Mph1103I, NlaIII, NsiI, NspI, PI-PspI, PaeI, Psp124BI, PsrI, PstI, RigI, SacI, SbfI, SdaI, SetI, SphI, Sse8387I, SstI, TaiI, TscAI, TspRI, XceI, Zsp2I. In some embodiments, the restriction enzyme capable of generating 3′ overhangs having a length of at least 4 nucleotides and having as 3′ terminal nucleotide A, T or G are those disclosed by Hoheisel (Analytical Biochemistry 1993; 209(2), 238-246). In certain embodiments, the restriction enzyme capable of generating 3′ overhangs having a length of at least 4 nucleotides and having as 3′ terminal nucleotide A, T or G is selected in the group consisting of AjuI, AloI, ArsI, BaeI, BarI, BplI, FalI and PsrI.

In practice, about 1 μg to about 5 μg of DNA nucleic acids may be treated with about 0.1u to about 10u of a suitable restriction enzyme capable of generating 3′ overhangs, as defined above. In practice, treatment with a polypeptide with a restriction enzyme capable of generating 3′ overhangs is or may be performed for about 30 min to about 180 min, preferably for about 60 min to about 120 min. In practice, treatment with a restriction enzyme capable of generating 3′ overhangs is or may be performed at a temperature of about 0° C. to about 45° C., preferably at a temperature of about 20° C. to about 40° C.

In some embodiments, step c) comprises recessing both ends of identical orientation of the linear nucleic acid of step a) or the nucleic acid obtained at step b1) or step b2), in the presence of a polypeptide having a 3′-5′ nuclease activity, so that HR1 and HR2 are capable of forming overlapping overhangs.

As used herein, the term “ends of identical orientation” refers either to the 3′ ends or to the 5′ ends, in particular the 3′ ends on each extremities of the double strand nucleic acid.

In practice, the assessment of a 3′-5′ nuclease activity may be performed by standard methods known in the state of the art, or a method adapted therefrom. Illustratively, the 3′-5′ nuclease activity may be assessed as disclosed in, e.g., Qiu et al. (J Biol Chem. 2005 Apr. 15; 280(15):15370-9), or with a commercial dedicated kit, such as, e.g., the BioVision's 3′ to 5′ Exonuclease Activity Assay Kit from BioVision®, following the manufacturer's instructions.

In some embodiments, the polypeptide having a 3′-5′ nuclease activity is Exonuclease III (also referred to as Exo III). In certain embodiments, Exo III is from E. coli. In practice, Exo III may be purchased commercially, e.g., from NEB® or Thermo Fisher Scientific®.

In practice, about 1 μg to about 10 μg, preferably about 1 μg to about 5 μg, of DNA nucleic acids may be treated with about 0.1u to about 100u of a polypeptide having a 3′-5′ nuclease activity, in particular Exo III. In practice, step c) is or may be performed for about 1 mM to about 20 mM, preferably for about 2 mM to about 15 min. In practice, step c) is or may be performed at a temperature of about 0° C. to about 37° C., preferably at a temperature of about 0° C. to about 35° C., preferably at a temperature of about 0° C. to about 25° C. In practice, step c) may be performed on ice or alternatively at a temperature of about 16° C. to about 30° C.

As used herein, 1u (1 unit) may be defined as the amount of the enzyme that catalyzes the release of 1 nmol of acid soluble reaction products from E. coli [³H]-DNA in 30 mM at 37° C.; the enzyme activity being assayed in a mixture comprising 50 mM Tris-HCl (pH 8.0), 5 mM MgCl₂, 1 mM DTT and 0.05 mM sonicated E. coli [³H]-DNA.

In some embodiments, the polypeptide having a 3′-5′ nuclease activity is a DNA polymerase with 3′-5′ nuclease activity. Illustratively, T4 DNA polymerase is one example of a DNA polymerase with 3′-5′ nuclease activity.

It is understood that at the end of step c) HR1 and HR2 are capable of forming overlapping overhangs. In other words, HR1 and HR2 form overhangs which are capable of overlapping.

In the embodiments wherein the linear double stranded DNA nucleic acid to be circularized is an amplicon obtained by PCR, a column purification kit may provide a mean to remove primers, dNTP and double-stranded primer dimers. In practice, suitable kits are, e.g., the PCR clean-up NucleoSpin® kits from Macherey-Nagel®.

In the embodiments wherein the linear double stranded DNA nucleic acid to be circularized is an amplicon obtained by PCR, an enzymatic PCR cleanup may provide an easy way to remove the remaining primers, dNTP and double-stranded primer dimers left from a PCR reaction.

In some embodiments, step b) or step c) is preceded by, or concomitantly performed with, a step comprising incubating the linear double stranded DNA nucleic acid of step a) with an alkaline phosphatase and/or a polypeptide with a type I exonuclease activity.

In certain embodiments, the alkaline phosphatase is the Shrimp Alkaline Phosphatase (SAP), in particular recombinant SAP (rSAP). rSAP is known to dephosphorylate the remaining dNTP. In practice, about 1 μg to about 5 μg of DNA nucleic acids may be treated with about 0.5u to about 5u of alkaline phosphatase. In practice, treatment with alkaline phosphatase is or may be performed for about 1 mM to about 20 mM, preferably for about 2 mM to about 15 mM In practice, treatment with alkaline phosphatase is or may be performed at a temperature of about 0° C. to about 45° C., preferably at a temperature of about 30° C. to about 40° C., preferably a temperature of about 37° C.

As used herein, 1u (1 unit) may be defined as the amount of enzyme which catalyzes the hydrolysis of 1 μmol of p-nitrophenyl phosphate per min in glycine buffer (pH 10.4) at 37° C.; the enzyme activity being assayed in a mixture comprising 100 mM glycine, pH 10.4, 1 mM MgCl₂, 1 mM ZnCl₂, 10 mM p-nitrophenyl phosphate.

In some embodiments, the polypeptide with a type I exonuclease activity is Exonuclease I (also referred to as Exo I). Exo I is known to degrade the residual PCR primers. In some embodiments, Exo I is a thermolabile Exo I. in certain embodiments, the Exo I is from E. coli. In practice, Exo I may be purchased from NEB®.

In practice, about 1 μg to about 5 μg of DNA nucleic acids may be treated with about 0.5u to about 25u of a polypeptide with a type I exonuclease activity. In practice, treatment with a polypeptide with a type I exonuclease activity is or may be performed for about 1 mM to about 20 min, preferably for about 2 mM to about 15 mM. In practice, treatment with a polypeptide with a type I exonuclease activity is or may be performed at a temperature of about 0° C. to about 45° C., preferably at a temperature of about 30° C. to about 40° C., preferably a temperature of about 37° C.

As used herein, 1u (1 unit) may be defined as the amount of enzyme that will catalyze the release of 2 nmol of acid-soluble nucleotide in a total reaction volume of 100 μl in 6 minutes at 37° C. with 0.17 mg/ml single-stranded [³H]-E.coli DNA; the enzyme activity being assayed in a mixture comprising 100 mM NaCl, 50 mM Tris-HCl, 10 mM MgCl₂, 100 μg/ml BSA, pH 7.9 at 25° C.

In some embodiments, the hairpins resulting from hybridization of NHR1-SOR1ₓ-HR1 and HR2-SOR2_{y}-NHR2, on one hand, or NHR1-SOR1ₓ-HR1-TBC and TBC-HR2-SOR2_{y}-NHR2, on the other hand, upon step b) contain less than 6 nucleotides annealed and are stable at less than about 35° C.

In practice, step d) is or may be performed by first bringing the reaction mixture at a temperature of about 70° C. to about 90° C., for about 1 min to about 10 mM, preferably for about 5 min, and then by bringing the reaction mixture at a temperature of about 45° C. to about 65° C., preferably at a temperature of about 50° C. to about 60° C., for about 5 mM to about 20 mM, preferably for about 10 min. A temperature of about 70° C. to about 90° C. allows to deactivate the mesophilic enzymes of the step b) and step c) by thermal treatment and to denature the single stranded ends of double stranded DNA nucleic acid to be circularized in order to release the overlapping overhangs. A temperature of about 45° C. to about 65° C. allows simultaneously to bend the dsDNA molecules with thermodynamic force and to anneal the single stranded overlapping overhangs within the same DNA molecule in order to obtain intramolecularly annealed dsDNA.

Within the scope of the instant invention, the expression “of about 70° C. to about 90° C.” encompasses about 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C. and 90° C. As used herein, the expression “for about 1 min to about 10 min” encompasses 1 min, 2 min, 3 min, 4 min, 5 min, 6 min, 7 min, 8 min, 9 min and 10 min. Within the scope of the instant invention, the expression “of about 45° C. to about 65° C.” encompasses about 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C. and 65° C. As used herein, the expression “for about 5 min to about 20 min” encompasses about 5 min, 6 min, 7 min, 8 min, 9 min, 10 min, 11 min, 12 min, 13 min, 14 min, 15 min, 16 min, 17 min, 18 min, 19 min and 20 min.

In some embodiments, the step d) may be performed by decreasing progressively the temperature of the reaction mixture from a first temperature of about 70° C. to about 90° C. to a second temperature of about 45° C. to about 65° C.

Within the scope of the instant invention, the term “decreasing progressively the temperature” is intended to mean that the difference of 2 temperatures is at least 1° C., preferably 2° C. and more preferably 5° C. Illustratively, decreasing progressively the temperature of the reaction mixture from a first temperature of, e.g., about 85° C. to a second temperature of about 45° C., may be performed by decreasing the temperature by 5° C.-20° C. at a time, i.e., by incubating the reaction mixture as follows:
85° C. for 1 min to 10 min;
65° C. for 1 min to 20 min, preferably for 5 min to 20 min;
60° C. for 1 min to 20 min, preferably for 5 min to 20 min;
55° C. for 1 min to 20 min, preferably for 5 min to 20 min;
50° C. for 1 min to 20 min, preferably for 5 min to 20 min;
45° C. for 1 min to 20 min, preferably for 5 min to 20 min.

In some embodiments, step d) is performed in the presence of divalent and/or trivalent cations.

In said embodiments, the divalent and/or trivalent cations provide annealing conditions favorable for hybridization of the HR1 and HR2 nucleic acids.

Non-limitative examples of divalent cations include calcium (Ca²⁺), magnesium (Mg²⁺) and manganese (Mn²⁺) cations. In some embodiments, the divalent cation is magnesium cation. In some embodiments, the final concentration of the divalent cations, in particular magnesium (Mg²⁺), in the reaction mixture ranges from about 0.01 mM to about 100 mM, preferably from about 10 to about 50 mM, more preferably from about 25 mM to about 30 mM. In certain embodiments, the divalent cation is magnesium cation. In some embodiments, the final concentration of the divalent cations, in particular magnesium (Mg²⁺), in the reaction mixture ranges from about 0.2 mM to about 20 mM. In some embodiments, the magnesium cations are provided in the form of a magnesium salt, in particular magnesium acetate or magnesium chloride.

Within the scope of the invention, the expression “about 0.01 mM to about 100 mM” includes about 0.01 mM, 0.02 mM, 0.04 mM, 0.06 mM, 0.08 mM, 0.1 mM, 0.2 mM, 0.4 mM, 0.6 mM, 0.8 mM, 1 mM, 1.2 mM, 1.4 mM, 1.6 mM, 1.8 mM, 2 mM, 2.5 mM, 3 mM, 3.5 mM, 4 mM, 4.5 mM, 5 mM, 5.5 mM, 6 mM, 6.5 mM, 7 mM, 7.5 mM, 8 mM, 8.5 mM, 9 mM, 9.5 mM, 10 mM, 11 mM, 12 mM, 13 mM, 14 mM, 15 mM, 16 mM, 17 mM, 18 mM, 19 mM, 20 mM, 22 mM, 24 mM, 26 mM, 28 mM, 30 mM, 32 mM, 34 mM, 36 mM, 38 mM, 40 mM, 42 mM, 44 mM, 46 mM, 48 mM, 50 mM, 55 mM, 60 mM, 65 mM, 70 mM, 75 mM, 80 mM, 85 mM, 90 mM, 95 mM and 100 mM.

In certain embodiments, the final concentration of the divalent cations, in particular magnesium (Mg²⁺), in the reaction mixture ranges from about 5 mM to about 15 mM, preferably from about 10 mM to about 12 mM.

Non-limitative examples of trivalent cations include aluminum (Al³⁺), chromium (Cr³⁺) and iron III (Fe³⁺) cations.

In some embodiments, step d) is performed at a pH ranging from about 6.0 to about 10.0, preferentially ranging from about 7.5 to about 8.5, at 25° C. Within the scope of the invention, the expression “about 6.0 to about 10.0” includes 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9 and 10.0.

In certain embodiments, step d) is performed in the presence of monovalent cations. Non-limitative examples of monovalent cations include silver (Ag⁺), copper (Cu⁺), lithium (Li⁺), potassium (K⁺) and sodium (Na⁺) cations.

In some embodiments, the final concentration of the monovalent cations in the reaction mixture ranges from about 0 mM to about 150 mM, preferably from about 0 mM to about 100 mM. Within the scope of the invention, the expression “about 0 mM to about 150 mM” includes 0 mM, 0.1 mM, 0.2 mM, 0.3 mM, 0.4 mM, 0.5 mM, 0.6 mM, 0.7 mM, 0.8 mM, 0.9 mM, 1 mM, 1.5 mM, 2 mM, 2.5 mM, 3 mM, 3.5 mM, 4 mM, 4.5 mM, 5 mM, 5.5 mM, 6 mM, 6.5 mM, 7 mM, 7.5 mM, 8 mM, 8.5 mM, 9 mM, 9.5 mM, 10 mM, 11 mM, 12 mM, 13 mM, 14 mM, 15 mM, 16 mM, 17 mM, 18 mM, 19 mM, 20 mM, 22 mM, 24 mM, 26 mM, 28 mM, 30 mM, 32 mM, 34 mM, 36 mM, 38 mM, 40 mM, 42 mM, 44 mM, 46 mM, 48 mM, 50 mM, 55 mM, 60 mM, 65 mM, 70 mM, 75 mM, 80 mM, 85 mM, 90 mM, 95 mM, 100 mM, 105 mM, 110 mM, 115 mM, 120 mM, 125 mM, 130 mM, 135 mM, 140 mM, 145 mM and 150 mM.

In some embodiments, the step d) is performed in the presence of one or more additional compound(s), such as, e.g., BSA, DTT, Tween, Tris, and the like. In said embodiments, the one or more additional compound(s) is/are intended to promote, favorize, annealing of HR1 and HR2 nucleic acids. The compounds of the buffer favor efficient enzymatic reactions at appropriate conditions.

In certain embodiments, the final concentration in the reaction mixture of the additional compound(s), such as, e.g., BSA, DTT, Tween, Tris, and the like, in particular Tris, ranges from about 5 mM to about 150 mM, preferably from about 50 mM to about 100 mM, more preferably from about 70 mM to about 80 mM. Within the scope of the invention, the expression “about 5 mM to about 150 mM” includes 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 22 mM, 24 mM, 26 mM, 28 mM, 30 mM, 32 mM, 34 mM, 36 mM, 38 mM, 40 mM, 42 mM, 44 mM, 46 mM, 48 mM, 50 mM, 55 mM, 60 mM, 65 mM, 70 mM, 75 mM, 80 mM, 85 mM, 90 mM, 95 mM, 100 mM, 105 mM, 110 mM, 115 mM, 120 mM, 125 mM, 130 mM, 135 mM, 140 mM, 145 mM and 150 mM.

A non-limitative example of reaction mixture to perform step d) comprises 25 mM potassium acetate, 10 mM Tris-acetate, 5 mM magnesium acetate, 100 μg/ml BSA, pH 7.9, at 25° C.

In some embodiments, step c) and step d) may be performed concomitantly. As used herein, the term “concomitantly” is intended to mean that the steps are performed in the same vessel. In said embodiments, the ingredients of the reaction mixture are added in the same vessel and the reaction conditions (temperature and time) of step c) and step d) are as mentioned above.

In some embodiments, step c) and step d) may be followed by treating the reaction mixture at a temperature comprised from about 0° C. to about 37° C., preferably a temperature of about 0° C. to about 10° C., preferably a temperature of about 2° C. to about 25° C., preferably a temperature of about 2° C. to about 5° C., for at least 1 mM. Within the scope of the instant invention, the term “about 0° C. to about 37° C.” includes 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C. and 37° C. As used herein, the term “at least 1 min” includes 1 mM, 2 mM, 3 mM, 4 mM, 5 mM, 6 mM, 7 mM, 8 mM, 9 mM, 10 mM, 15 mM, 20 mM, or longer.

By the end of step d), 2 gaps, 2 nicks, or 1 gap and 1 nick are generated. At the end of the step d) intramolecularly annealed dsDNA, i.e., a circular tri-dimensional structure of DNA, is obtained. In practice, 2 gaps are generated when TBC comprises 2 sites of nicking restriction on different DNA strands each located about 1 nucleotide to about 100 nucleotides respectively from the 3′ end of HR1 and from the 3′ end of HR2. In practice, 2 nicks are generated when TBC comprises 2 sites of nicking restriction on different DNA strands each located 0 nucleotide respectively from the 3′ end of HR1 and from the 3′ end of HR2.

It some embodiments, step e) is optional when 2 nicks are generated on one strand at step d).

In certain embodiments, step e) is performed in the presence of a DNA polymerase with no strand displacement activity and with no 5′ to 3′ exonuclease activity and dNTPs and/or of oligonucleotides with 5′-phosphate having a length of about 8 nucleotides to about 100 nucleotides and being complementary to the nucleic acids of the gaps, provided the polypeptide having a 3′-5′ nuclease activity has been inactivated.

In practice, when x and y are 0, step e) is performed in the presence of a DNA polymerase and dNTPs, so as to fill the gaps. In said embodiment, 2 nicks are generated.

Alternatively, when x and y are 0, step e) may be performed in the presence of oligonucleotides and in the presence of a DNA polymerase and dNTPs. In said embodiment, more than 2 nicks are generated.

In some embodiments, the DNA polymerase with no strand displacement activity and with no 5′ to 3′ exonuclease activity is the T4 DNA polymerase. In practice, T4 DNA polymerase may be purchased from Promega®, NEB®, Takara®, Thermo Fisher Scientific®.

In practice, about 1 μg to about 5 μg of DNA nucleic acids may be treated with about 1u to about 50u of a DNA polymerase. In practice, step e) is or may be performed for about 10 mM to about 120 mM, preferably for about 30 mM to about 90 mM. In practice, step e) is or may be performed at a temperature of about 0° C. to about 40° C., preferably at a temperature of about 10° C. to about 25° C., preferably at a temperature of about 15° C. to about 25° C., preferably a temperature of about 20° C.

As used herein, 1u (1 unit) of the enzyme catalyzes the incorporation of 10 nmol of deoxyribonucleotides into a polynucleotide fraction in 30 mM at 37° C.

As used herein, dNTPs include dATP, dCTP, dTTP and dGTP. In practice, step e) is or may be performed in the presence of about 0.05 mM to about 15 mM of dNTPs, preferably in the presence of about 0.05 mM to about 10 mM of dNTPs, more preferably in the presence of about 1 mM to about 5 mM of dNTPs.

In practice, when x and y are 1, step e) is performed in the presence of a DNA polymerase and dNTPs, and/or in the presence of oligonucleotides, so as to fill the gaps. In said embodiments, the length of the gaps would depend of the location of the nicks generated by the polypeptide with nicking activity. In said embodiments, the oligonucleotides are complementary to the nucleic acids from the gaps.

In practice, the oligonucleotides may have a length of about 8 nucleotides to about 100 nucleotides. In some embodiments, the oligonucleotides have a length of about 18 nucleotides to about 30 nucleotides.

Within the scope the instant invention, the expression “about 8 nucleotides to about 100 nucleotides” includes 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98 and 100 nucleotides.

In certain embodiments, when the gaps have a length of about 1 nucleotide to 8 nucleotides, step e) is advantageously performed in the presence of a DNA polymerase and dNTPs. In said embodiments, step e) is performed in the absence of oligonucleotides.

In practice, the presence of a polypeptide having 3′-5′ endonuclease activity, in particular ExoIII in the reaction mixture, may digest oligonucleotides as soon as they anneal to the DNA nucleic acid at room temperature. Therefore, in some embodiments, inactivation of the polypeptide having 3′-5′ nuclease activity, in particular ExoIII, may be performed.

In practice, inactivation of the polypeptide having 3′-5′ nuclease activity, in particular ExoIII, may be performed by thermal inactivation. In some embodiments, thermal inactivation may comprise heating and cooling of the reaction mixture. In practice, heating of the reaction mixture may be performed at a temperature ranging from about 70° C. to about 90° C., during about 5 min to about 10 min.

Within the scope of the invention, the term “about 70° C. to about 90° C.” includes 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C. and 90° C. Within the scope of the invention, the term “about 5 min to about 10 min” includes 5, 6, 7, 8, 9 and 10 min.

It is understood that the method disclosed herein may be performed exclusively in vitro, i.e., without the requirement of any living organism, such as, e.g., a bacterium.

However, in some embodiments, step e) may be performed by a competent bacterium. In practice, the nucleic acid obtained at the end of step d) may be introduced in a competent bacterium, by any suitable method known in the state of the art, or a method adapted therefrom, in particular transformation. In said embodiment, the method is hence performed substantially in vitro. As used herein, the term “substantially in vitro” is intended to mean that at most one step of the method is performed within a living organism, hence in vivo.

The term “transformation” is used herein to refer to the introduction of an extra-genomic nucleic acid in the cytoplasm of a bacterium. The bacterium, the cytoplasm of which contains at least one extra-genomic nucleic acid after the step of transformation, are qualified as “transformed bacterium”. The transformation of bacteria requires typically that said cells have been beforehand treated to become “competent” for the extra-genomic nucleic acid to enter the cytoplasm upon transformation. As used herein, the term “competent” refers to a bacterium that has an increased ability to uptake an extra genomic nucleic acid into its cytoplasm. The skilled artisan is familiar with techniques for preparing competent bacteria. Illustratively, for the steps of preparation of competent bacteria, transformation, selection of transformed bacteria one may refer to the manufacturer's instructions, when commercial kits or materials are used, and/or refer to, e.g., the protocols described by J. Sambrook and D. Russell, Molecular Cloning: A Laboratory Manual, 3^{rd} ed., Cold Spring Harbor Laboratory Press, Cold Spring Harbor, N. Y. (2001).

In some embodiments, competent bacteria may be chemically competent cells, in particular calcium chloride-treated bacteria. In some alternative embodiments, competent bacteria may be electrocompetent bacteria. In practice, chemically competent or electrocompetent bacteria may be purchased from Thermo Fisher Scientific®, Sigma-Aldrich® or NEB®. For E. coli bacteria, a non-limitative list of commercial chemically competent bacteria encompasses BL21(DE3), DH10B, DH5α, Mach1, TOP10, INV110, SIG10. A non-limitative list of commercial E. coli electrocompetent bacteria encompasses Mega DH10B T1R, ElectroMAX DH5α, One shot TOP10, SIG10 MAX.

The optional step f) of removing the NHR1 and NHR2 nucleic acids at both 5′ ends, when the length of the NHR1 and NHR2 nucleic acids is superior to 0 bp is performed when x and y are 0, i.e. when SOR1 and SOR2 are absent. In said step, the single stranded 5′ overhangs generated by the 3′-5′ nuclease are recessed.

In some embodiments, step f) is performed in the presence of a polypeptide having a 5′-3′ nuclease activity, preferably Exonuclease VII (Exo VII), RecJ_{f} or Flap endonuclease 1 (FEN1).

In practice, the assessment of a 5′-3′ nuclease activity may be performed by standard methods known in the state of the art, or a method adapted therefrom. Illustratively, the 5′-3′ nuclease activity may be assessed as disclosed in, e.g., Lyamichev et al. (PNAS. 1999 May 25; 96(11):6143-8), Amblar et al. (J Biol Chem. 2001 Jun. 1; 276(22):19172-81). In practice, ExoVII, RecJ_{f} or FEN1 nucleases may be purchased from, e.g., NEB®, Thermo Fisher Scientific®.

In practice, about 1 μg to about 5 μg of DNA nucleic acids may be treated with about 0.01u to about 10u of a polypeptide having a 5′-3′ nuclease activity. In practice, step is or may be performed for about 30 mM to about 120 mM, preferably for about 45 min to about 90 min. In practice, step f) is or may be performed at a temperature of about 0° C. to about 37° C., preferably at a temperature of about 15° C. to about 25° C., preferably a temperature of about 20° C.

As used herein, 1u (1 unit) of ExoVII may be defined as the amount of enzyme that will catalyze the release of 1 nmol of acid-soluble nucleotide in a total reaction volume of 50 μl in 30 minutes at 37° C. As used herein, 1u (1 unit) is defined as the amount of RecJ_{f} required to produce 0.05 nmol TCA soluble deoxyribonucleotide in a total reaction volume of 50 μl in 30 minutes at 37° C. in a suitable buffer 50 mM NaCl, 10 mM Tris-HCl, 10 mM MgCl₂, 1 mM DTT, pH 7.9 at 25° C.) with 1.5 μg sonicated single-stranded [³H]-labeled E. coli DNA. As used herein, 1u (1 unit) of FEN1 may be defined as the amount of FEN1 required to cleave 10 pmol of 5′ DNA flap containing oligonucleotide substrate in a total reaction volume of 10 μl for 10 minutes at 65° C.

In some embodiments, step g) is performed in the presence of a mesophilic DNA ligase, preferably T4 DNA ligase.

In practice, T4 DNA ligase may be purchased from, e.g., Thermo Fisher Scientific®, NEB®, Invitrogen®, Takara®. In some embodiments, the DNA ligase is a salt resistant DNA ligase. In practice, the salt resistant DNA ligase may be purchased from NEB® (Salt-T4® DNA ligase).

In practice, about 1 μg to about 5 μg of DNA nucleic acids may be treated with about 1u to about 500u of a DNA ligase. In practice, step g) is or may be performed for about 15 min to about 120 min, preferably for about 45 min to about 90 min. In practice, step g) is or may be performed at a temperature of about 0° C. to about 37° C., preferably at a temperature of about 15° C. to about 25° C.

As used herein, 1u (1 unit) catalyzes the exchange of 1 nmol [³²P]-labeled pyrophosphate into ATP in 20 min at 37° C.; the enzymatic activity being assayed in a mixture comprising 66 mM Tris-HCl (pH 7.6), 6.6 mM MgCl₂, 10 mM DTT, 66 μM ATP, 3.3 μM [³²P]-labeled pyrophosphate.

In some embodiments, step g) is performed in the presence of ATP. In practice, step g) is or may be performed in the presence of about 0.05 mM to about 15 mM of ATP, preferably in the presence of about 0.1 mM to about 10 mM of ATP, more preferably in the presence of about 1 mM to about 5 mM of ATP.

Within the scope of the invention, the term “about 0.05 mM to about 15.0 mM” includes 0.05 mM, 0.10 mM, 0.15 mM, 0.20 mM, 0.25 mM, 0.30 mM, 0.35 mM, 0.40 mM, 0.45 mM, 0.50 mM, 0.55 mM, 0.60 mM, 0.65 mM, 0.70 mM, 0.75 mM, 0.80 mM, 0.85 mM, 0.90 mM, 0.95 mM, 1.0 mM, 1.5 mM, 2.0 mM, 2.5 mM, 3.0 mM, 3.5 mM, 4.0 mM, 4.5 mM, 5.0 mM, 5.5 mM, 6.0 mM, 6.5 mM, 7.0 mM, 7.5 mM, 8.0 mM, 8.5 mM, 9.0 mM, 9.5 mM, 10.0 mM, 10.5 mM, 11.0 mM, 11.5 mM, 12.0 mM, 12.5 mM, 13.0 mM, 13.5 mM, 14.0 mM, 14.5 mM and 15.0 mM.

In some embodiments, step g) is performed in the absence of any polymer, in particular in the absence of polyethylene glycol (PEG). Said characteristic is advantageous as compared to numerous nucleic acids assembly methods, such as, e.g., those described by US patent n° 8,968,999 (Gibson et al.) as well as those described by Chen Cheng et al. (J Med Genet 2019; 56:10-17), which require the presence of PEG, in particular PEG 4000.

In some embodiments, step e), step f) and step g) may be performed concomitantly.

In practice, the presence of circularized double stranded DNA nucleic acids may be assessed with any suitable method known in the state of the art, or a method adapted therefrom. Illustratively, the products of a circularization method according to the invention may be analyzed on an agarose gel. Circularized double stranded DNA nucleic acids would appear heavier than the corresponding linear double stranded DNA nucleic acids.

It is understood that the method may be performed in vitro, i.e. without the requirement of a living organism, in particular, a bacterium.

In some embodiments, step g) may be performed by a competent bacterium. In practice, the nucleic acid obtained at the end of step f) may be introduced in a competent bacterium, by any suitable method known in the state of the art, or a method adapted therefrom, in particular transformation.

In certain embodiments, when NHR1 and NHR2 represent nucleic acids of 0 bp, step f) is not performed, and step e) and step g) may be performed by a competent bacterium. In practice, the nucleic acid obtained at the end of step d) may be introduced in a competent bacterium, by any suitable method known in the state of the art, or a method adapted therefrom, in particular transformation.

In some embodiments, both strands of the circularized nucleic acid are circular. In said embodiments, all the nicks generated within the course of the method are sealed.

In certain embodiments, said method may further comprise the step of:
h) removing the linear and/or non-circularized DNA nucleic acids.

In practice this step allows removing both linear and/or non-circularized double-stranded and single-stranded DNA molecules. Illustratively, this step may be performed in the presence of a polypeptide having an exonuclease activity, such as, e.g., Exonuclease V (also referred to as Exo V or RecBCD), truncated Exonuclease VIII (also referred to as truncated Exo VIII), T7 exonuclease or Lambda Exonuclease. In practice, when step h) is performed in the presence of Exo V, ATP is further included in the reaction mixture. In practice, Exo V, truncated Exo VIII or Lambda Exonuclease may be purchased from NEB® or Thermo Fisher Scientific®.

In practice, about 1 μg to about 5 μg of DNA nucleic acids may be treated with about 1u to about 100u of Exo V, truncated Exo VIII or Lambda Exonuclease. In practice, step h) is or may be performed for about 15 mM to about 120 mM, preferably for about 45 min to about 90 mM In practice, step h) is or may be performed at a temperature of about 0° C. to about 45° C., preferably at a temperature of about 30° C. to about 40° C., preferably at a temperature of about 37° C.

One unit is defined as the amount of Exo V required to produce 1 nmol of acid-soluble deoxyribonucleotide from double stranded DNA in 30 minutes at 37° C. in a total reaction volume of 50 μl; the enzyme activity being assayed in a mixture comprising 1 mM ATP and 50 mM Potassium Acetate, 20 mM Tris-acetate, 10 mM Magnesium Acetate, 1 mM DTT, pH 7.9 at 25° C.

1u (one unit) is defined as the amount of Lambda Exonuclease required to produce 10 nmol of acid-soluble deoxyribonucleotide from double-stranded substrate in a total reaction volume of 50 μl in 30 minutes at 37° C. in the suitable buffer (67 mM Glycine-KOH, 2.5 mM MgCl₂, 50 μg/ml BSA, pH 9.4 at 25° C.), with 1 μg sonicated duplex [³H]-DNA.

In some embodiments, step h) may be followed by treating the reaction mixture with EDTA, preferably at a final concentration ranging from about 1 mM to about 25 mM, more preferably at a final concentration ranging from about 5 mM to about 15 mM, and/or at a temperature ranging from about 60° C. to about 80° C., for 15 mM to 60 min. Within the scope of the instant invention, the term “about 1 mM to about 25 mM” includes 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM and 25 mM.

In certain embodiments, the method further includes a step of binding the double stranded DNA nucleic acid, in particular the circularized double stranded DNA nucleic acid, to one or more non-nucleic acid moiety (moieties), preferably selected in the group comprising linkers, polypeptides, particles, surfaces, and combinations thereof; so as to generate a functionalized binding of the double stranded DNA nucleic acid, in particular a functionalized circularized double stranded DNA nucleic acid.

As used herein, the term “binding” encompasses both covalent and non-covalent binding.

In practice, covalent binding may also refer to as “conjugation”.

In some embodiments, this step is a functionalization step. In practice, functionalization may be performed at various stages of the method.

In some embodiments, linkers, polypeptides may be introduced during PCR amplification, so as to obtain a functionalized linear nucleic acid of formula (I).

In practice, functionalization may be performed by the means of one or more modified primer(s) capable of introducing said linker and/or said polypeptide into the HR1 and/or HR2 or TBC. Alternatively, functionalization may also be performed during step d), by the mean of modified dNTP nucleotides, or by the mean of modified oligonucleotides. Alternatively, functionalization may be performed as a late stage of the method, i.e., onto a circularized double stranded DNA nucleic acid according to the invention.

In some embodiments, step e) further includes annealing of one or two or several oligonucleotides, preferentially modified with one to ten polypeptides and/or one to ten linkers, with the intramolecularly annealed dsDNA molecule providing one or two gaps. The site or the sites for the oligonucleotide annealing are localized within gap region of TBC. The length and the sequence of the oligonucleotide assures specific annealing, as it is well known in the state of the art, and may range from 8 nucleotides to 100 nucleotides, preferentially from 18 nucleotides to 30 nucleotides.

In practice, the said functional polypeptides are well known in the state of the art, and are employed to facilitate the penetration of nucleic acid molecules into a target recipient cell, to facilitate the nuclear localization of the nucleic acid molecules into a target recipient cell, to facilitate the localization in cell organelles of the nucleic acid molecules into a target recipient cell, and/or to facilitate the binding to epitopes of choice on cell surface.

In some embodiments, the polypeptide facilitating the penetration into a target recipient cell may comprise, or consist of, a cell-penetrating peptide (CPP), also referred to as “protein transduction domain” (PTD). As used herein, cell-penetrating peptides refer to peptides being generally short in length, i.e., of up to 30 residues, having a net positive charge and acting in a receptor-independent and energy-independent manner.

In certain embodiments, the functional polypeptide according to the invention may comprise one or more CPPs. In said embodiments, the CPP may advantageously be cleavable upon penetration into a recipient cell. Examples of CPPs include those selected in the group consisting of hydrophilic CPPs, hydrophobic CPPs, and amphipathic CPPs. In practice, hydrophilic CPPs are peptides composed mainly by hydrophilic amino acids usually rich in amino acid residues R and K, whereas amphipathic CPPs are peptides that contain an alternating pattern of polar/charged amino acids and non-polar, hydrophobic amino acids, and are usually rich in amino acid residue K. On the other hand, hydrophobic CPPs are peptides containing only apolar residues, with low net charge or have hydrophobic amino acid groups that are crucial for cellular uptake.

In some embodiments, hydrophilic CPPs may be selected in the non-limitative group comprising Antennapedia Penetratin (RQIKWFQNRRMKWKK, SEQ ID NO. 1), BMV Gag-(7-25) (KMTRAQRRAAARRNRWTAR, SEQ ID NO. 2), D-Tat (GRKKRRQRRRPPQ, SEQ ID NO. 3), FHV Coat-(35-49) (RRRRNRTRRNRRRVR, SEQ ID NO. 4), HTLV-II Rex-(4-16) (TRRQRTRRARRNR, SEQ ID NO. 5), PTD-4 (PIRRRKKLRRLK, SEQ ID NO. 6), PTD-5 (RRQRRTSKLMKR, SEQ ID NO. 7), R9-Tat (GRRRRRRRRRPPQ, SEQ ID NO. 8), SynB1 (RGGRLSYSRRRFSTSTGR, SEQ ID NO. 9), SynB3 (RRLSYSRRRF, SEQ ID NO. 10), and TAT (YGRKKRRQRRR, SEQ ID NO. 11).

The antennapedia-derived penetratin (Derossi et al. (1994)) and the Tat peptide (Vives et al. (1997)), or their derivatives, are in particular widely used tools for the delivery of cargo molecules such as peptides, proteins and oligonucleotides into cells (Fischer et al. (2001)).

In some embodiments, amphipathic CPPs may be selected in the non-limitative group comprising Transportan (GWTLNSAGYLLGKINLKALAALAKKIL, SEQ ID NO. 12), MAP (KLALKLALKLALALKLA, SEQ ID NO. 13), SBP (MGLGLHLLVLAAALQGAWSQPKKKRKV, SEQ ID NO. 14), FBP (GALFLGWLGAAGSTMGAWSQPKKKRKV, SEQ ID NO. 15), MPG (GALFLGFLGAAGSTMGAWSQPKKKRKV, SEQ ID NO. 16), MPG^{(ΔNLS)} (GALFLGFLGAAGSTMGAWSQPKSKRKV, SEQ ID NO. 17), Pep-1 (KETWWETWWTEWSQPKKKRKV, SEQ ID NO. 18), and Pep-2 (KETWFETWFTEWSQPKKKRKV, SEQ ID NO. 19).

In certain embodiments, the CPP may be comprised in a sequence selected in a group comprising SEQ ID NO. 1 to SEQ ID NO. 19.

In some embodiments, the CPPs may be such as those disclosed in the patent applications WO 2011/157713 and WO 2011/157715 (Hoffmann La Roche®), or derivatives thereof.

In some embodiments, the polypeptide facilitating the penetration into a target recipient cell may comprise, or consist of, a nuclear localization domain. In practice, suitable classical or non-classical nuclear localization domains may be such as those disclosed in, e.g., Lange et al. (2007), Kosugi et al. (2009) and Marfori et al. (2011).

Illustratively, the nuclear localization domain may be selected in the non-limitative group comprising the sequences PAAKRVKLD (SEQ ID NO. 20) of c-Myc, MSRRRKANPTKLSENAKKLAKEVEN (SEQ ID NO. 21) of EGL-13, PAATKKAGQA (SEQ ID NO. 22) or KRPAATKKAGQAKKKK (SEQ ID NO. 23) of nucleoplasmin, and PKKKRKV (SEQ ID NO. 24) of SV40.

In certain embodiments, the nuclear localization domain may be comprised in a sequence selected in a group comprising SEQ ID NO. 20 to SEQ ID NO. 24.

In some embodiments, the polypeptide facilitating the penetration into a target recipient cell may comprise, or consist of, an organelle localization domain.

As used herein, the term “organelle” is intended to refer to a specific cellular compartment, such as, e.g., the chloroplast, the endoplasmic reticulum (ER), the mitochondria, the peroxisome, the vacuoles, and the like.

Illustratively, the sequence MMSFVSLLLVGILFWATEAEQLTKCEVFQ (SEQ ID NO. 25) may promote ER localization; the sequence MLSLRQSIRFFKPATRTLCSSRYLL (SEQ ID NO. 26) may promote mitochondria localization; the sequence MVAMAMASLQSSMSSLSLSSNSFLGQPLSPITLSPFLQG (SEQ ID NO. 27) may promote chloroplast localization; the sequences SKL and XXXXRLXXXXXHL (SEQ ID NO. 28), wherein X may represent any amino acid residue, may promote peroxisome localization; the sequences HSRFNPIRLPTTHEPA (SEQ ID NO. 29), SSSSFADSNPIRPVTDRAASTLE (SEQ ID NO. 30), VFAEAIAANSTLVAE (SEQ ID NO. 31), NGLLVDTM (SEQ ID NO. 32), VSGGVWDSSVETNATASLVSEM (SEQ ID NO. 33) and QAHPNFPLEMPGSDEVAK (SEQ ID NO. 34) may promote vacuole localization.

In certain embodiments, the organelle localization domain may be comprised in a sequence selected in a group comprising SEQ ID NO. 25 to SEQ ID NO. 34.

In certain embodiments, the double stranded DNA nucleic acid according to the invention may be covalently bound to the at least one functional polypeptide by the mean of a linker. As used herein, the term “linker” is intended to refer to an organic entity that covalently or non-covalently attaches the functional polypeptide to the double stranded nucleic acid of the invention. In practice the linker may comprise from 1 to 1,000 plural valent atoms selected from the group consisting of C, N, O, S and P. In practice, the linker may be linear or non-linear; and some linkers may have pendant side chains or pendant functional groups (or both).

In some embodiments, the linker may be a Gly-rich linker, in particular comprising one or more “GS” motifs, such as, e.g., GGSSG (SEQ ID NO. 35), GSGSGS (SEQ ID NO. 36), GGSGGSGGSGG (SEQ ID NO. 37), GGGGSLVPRGSGGGGS (SEQ ID NO. 38), GGSGGHMGSGG (SEQ ID NO. 39), SGGGSSHS (SEQ ID NO. 40), SGGSGGSSHS (SEQ ID NO. 41), and SGGSGGSGGSSHS (SEQ ID NO. 42). In certain embodiments, the linker may be biotin or streptavidin.

In some embodiments, the functional polypeptide may be preferentially bound to an oligonucleotide in order to purify it prior annealing and, next, the polypeptide-oligonucleotide conjugate may be annealed to the gap region of intramolecularly annealed dsDNA and sealed at the step g) with a mesophilic ligase.

In certain embodiments, the double stranded DNA nucleic acid according to the invention may be bound to, in particular, covalently bound to, one or more antibody/ies, fragment(s) thereof, afucosylated antibody/ies, diabody/ies, triabody/ies, tetrabody/ies, nanobody/ies, and analog(s) thereof.

As used herein, an “antibody”, also referred to as immunoglobulins (abbreviated “Ig”), is intended to refer to a gamma globulin protein that is found in blood or other bodily fluids of vertebrates, and is used by the immune system to identify and neutralize foreign objects, such as bacteria and viruses. Antibodies consist of two pairs of polypeptide chains, called heavy chains and light chains that are arranged in a Y-shape. The two tips of the Y are the regions that bind to antigens and deactivate them. The term “antibody” (Ab) as used herein includes monoclonal antibodies, polyclonal antibodies, multi-specific antibodies (e.g., bispecific antibodies), and antibody fragments, so long as they exhibit the desired biological activity. The term “immunoglobulin” (Ig) is used interchangeably with “antibody” herein.

As used herein, an “antibody fragment” comprises a portion of an intact antibody, preferably the antigen binding or variable region of the intact antibody. Examples of antibody fragments include Fab, Fab′, F(ab′)2, and Fv fragments; diabodies; linear antibodies (see U.S. Pat. No. 5,641,870; Zapata et al., Protein Eng. 8(10): 1057-1062) [1995]; single-chain antibody molecules; and multi-specific antibodies formed from antibody fragments. One may refer to a “functional fragment or analog” of an antibody, which is a compound having qualitative biological activity in common with a full-length antibody. For example, a functional fragment or analog of an anti-IgE antibody is one that can bind to an IgE immunoglobulin in such a manner so as to prevent or substantially reduce the ability of such molecule from having the ability to bind to the high affinity receptor, Fc[epsilon]RI. Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (CH1). Each Fab fragment is monovalent with respect to antigen binding, i.e., it has a single antigen-binding site. Pepsin treatment of an antibody yields a single large F(ab′)2 fragment that roughly corresponds to two disulfide linked Fab fragments having divalent antigen-binding activity and is still capable of cross-linking antigen. Fab′ fragments differ from Fab fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments that have hinge cysteines between them. Other chemical couplings of antibody fragments are also known.

As used herein, an “afucosylated antibody” refers to an antibody lacking core fucosylation. As a matter of fact, nearly all IgG-type antibodies are N-glycosylated in their Fc moiety. Typically, a fucose residue is attached to the first N-acetylglucosamine of these complex-type N-glycans. In other words, an “afucosylated antibody” refers to an antibody that does not possess N-glycans.

As used herein, the term “diabody” refers to a small antibody fragment prepared by constructing sFv fragments (see preceding paragraph) with short linkers (about 5-10 residues) between the VH and VL domains such that inter-chain but not intra-chain pairing of the V domains is achieved, resulting in a bivalent fragment, i.e., fragment having two antigen-binding sites. Bispecific diabodies are heterodimers of two “crossover” sFv fragments in which the VH and VL domains of the two antibodies are present on different polypeptide chains. Diabodies are described in more details in, e.g., EP 404,097; WO 93/11161; and Hollinger et al., Proc. Natl. Acad. Sci. USA, 90:6444-6448 (1993).

As used herein, a “triabody” is intended to refer to an antibody that has three Fv heads, each consisting of a VH domain from one polypeptide paired with the VL domain from a neighboring polypeptide.

As used herein, a “nanobody” refers to a functional antibody that consists of heavy chains only and therefore lack light chains. These heavy-chain only antibodies contain a single variable domain (VHH) and two constant domains (CH2, CH3).

In some embodiments, antibodies may be bound to the circularized DNA at the last step of the method according to the instant invention.

In some embodiments, the particles are nanoparticles, microparticles, or a combination thereof.

Circularized double stranded nucleic acids according to the invention may be bound to a surface, such as e.g., a vessel, a petri dish, a fabric, or a cellular surface.

In further aspects, the invention relates to a method, in particular an in vitro method, for the circularization and the functionalization of a double stranded DNA nucleic acid. In practice, the method comprises the steps as defined herein.

In some embodiments, x and y are 0, and the linear double stranded DNA nucleic acid comprises a nucleic acid of the formula (II): NHR1-HR1-TBC-HR2-NHR2 (II).

In said embodiments, the method for the circularization of a double stranded DNA nucleic acid comprises the steps of:
a) providing a linear or circular double stranded DNA nucleic acid comprising a nucleic acid of the following formula (II):

NHR1-HR1-TBC-HR2-NHR2 (II),

wherein:
TBC represents a core double stranded DNA nucleic acid to be circularized, in particular having a length of at least about 250 bp, preferably at least about 500 bp, more preferably at least about 1,000 bp;
HR1 and HR2 represent identical homologous double stranded DNA nucleic acids of identical orientation;
NHR1 and NHR2 represent distinct non-homologous double stranded DNA nucleic acids having a length of 0 bp to about 200 bp;
b) digesting the circular double stranded DNA nucleic acid comprising a nucleic acid of formula (II) from step a), in the presence of a restriction enzyme that cleaves a restriction site that is not located in any one of HR1, HR2 and TBC, so as to obtain a linearized nucleic acid;
c) recessing both ends of identical orientation of the linearized nucleic acid obtained in step b), in the presence of a polypeptide having a 3′-5′ nuclease activity;
d) annealing the DNA nucleic acid obtained at step c), thereby generating 2 gaps;
e) filling the 2 gaps;
f) optionally removing the NHR1 and/or NHR2 at one or both 5′ ends, when the length of the NHR1 and NHR2 is superior to 0 bp;
g) sealing at least one nick, so as to obtain a circularized double stranded DNA nucleic acid, in which at least one strand is continuous.

In some embodiments, x and y are 1, and the linear double stranded DNA nucleic acid comprises a nucleic acid of formula (III):

NHR1-SOR1-HR1-TBC-HR2-SOR2-NHR2 (III).

In said embodiments, the method for the circularization of a double stranded DNA nucleic acid comprises the steps of:

a) providing a linear or circular double stranded DNA nucleic acid comprising a nucleic acid of the following formula (III):

NHR1-SOR1-HR1-TBC-HR2-SOR2-NHR2 (III),
wherein:
   TBC is a core double stranded DNA nucleic acid to be circularized, in particular having a length of at least about 250 bp, preferably at least about 500 bp, more preferably at least about 1,000 bp; TBC further comprises 2 sites of nicking restriction each located about 0 nucleotide to about 100 nucleotides respectively from the 3′ end of HR1 and from the 3′ end of HR2;
   HR1 and HR2 represent identical homologous double stranded DNA nucleic acids of identical orientation;
   SOR1 and SOR2 represent nucleic acids having a length of about 5 bp to about 60 bp comprising a site of restriction capable of generating 3′ overhangs having a length of at least 4 nucleotides and having as 3′ terminal nucleotide A, T or G;
   NHR1 and NHR2 represent distinct non-homologous double stranded DNA nucleic acids having a length of 0 bp to about 200 bp;
b1) optionally digesting the circular double stranded DNA nucleic acid comprising a nucleic acid of formula (III) from step a), in the presence of a restriction enzyme that cleaves a restriction site that is not located in any one of HR1, HR2 and TBC, so that to generate a linearized nucleic acid;
b2) digesting the circular, linear or linearized nucleic acid in the presence of (i) a polypeptide with nickase activity capable of introducing a nick within TBC, and (ii) restriction enzyme(s) capable of digesting the corresponding site of restriction in SOR1 and SOR2;
c) recessing both ends of identical orientation of the nucleic acid of step b2) in the presence of a polypeptide having a 3′-5′ nuclease activity;
d) annealing the DNA nucleic acid obtained at step c), thereby generating 2 gaps, 2 nicks, or 1 gap and 1 nick;
e) filling the 1 or 2 gaps, step e) being optional when 2 nicks are generated at step d);
f) optionally removing the NHR1 and/or NHR2 at one or both 5′ ends, when the length of the NHR1 and NHR2 is superior to 0 bp
g) sealing at least one nick, so as to obtain a circularized double stranded DNA nucleic acid in which at least one strand is continuous.

In certain embodiments, when the linear double stranded nucleic acid of step a) comprises 5′-phospate ends and one or more thiophosphate nucleotide(s), the circularization may be performed in one single vessel, in the presence of at least one inhibitor of the Pfu DNA polymerase; a polypeptide having a 3′-5′ nuclease activity, in particular ExoIII; polypeptides having an exonuclease activity, in particular ExoI, and Lambda exonuclease; a DNA polymerase, in particular Hot-Start Taq DNA polymerase; a DNA ligase, in particular Taq ligase; dNTPS; ATP; and appropriate buffers.

In practice, the Lambda exonuclease recesses 5′ ends up to the first thiophosphate nucleotide it encounters. In other words, the Lambda exonuclease recesses 5′ ends but cannot recess thiophosphate bonds. In practice, exonuclease ExoI recesses 3′ ends up to a 5′-phosphate end, so as to provide blunt ends.

In practice, the inhibitor of the Pfu DNA polymerase may be any one of the compounds described in Sun et al. (Sci Rep 2018; 8:1990) or in Lasken et al. (J Biol Chem 1996; 271(30):17692-17696).

In practice, the 3′ ends of the linear double stranded nucleic acid of step a) are or may be recessed in the presence of ExoIII and ExoI, whereas the 5′ ends of the linear double stranded nucleic acid of step a) are recessed in the presence of Lambda exonuclease.

In these embodiments, the circularized double stranded DNA nucleic acid according to the invention comprises the one or more thiophosphate nucleotide(s), as defined above.

In practice, the S atom of the thiophosphate nucleotide(s) allows site-specific functionalization of the circularized double stranded DNA nucleic acid according to the invention.

In some embodiments, the circularized double stranded DNA nucleic acid according to the invention is exclusively from bacterial and/or viral origin. As used herein, the term “exclusively from bacterial and/or viral origin” is intended to mean that at least 75% of the circularized double stranded DNA nucleic acid according to the invention is from bacterial and/or viral origin. As used herein, the term “at least 75%” encompasses 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% and 100%. As used herein, the term “from bacterial and/or viral origin” is intended to mean that the circularized double stranded DNA nucleic acid comprises bacterial and/or viral DNA.

In certain embodiments, the circularized double stranded DNA nucleic acid according to the invention is free of bacterial and/or viral nucleic acid. In some embodiments, the circularized double stranded DNA nucleic acid as defined herein is at least 75% from human origin, preferably at least 80%, preferably at least 90%, more preferably at least 95% from human origin. In certain embodiments, the circularized double stranded DNA nucleic acid according to the invention is 100% from human origin. These embodiments are particularly advantageous because bacterial-free and/or viral-free circularized double stranded DNA nucleic acids are less prone to promote an immune response, in particular an innate immune response.

Advantageously, the above-described methods may be performed in less than about 3 h. In some embodiments, the method as defined herein may be performed in about 1 h30 to about 3 h00. As used herein, the term “about 1 h30 to about 3 h00” encompasses, about 1 h30, 1 h35, 1 h40, 1 h45, 1 h50, 1 h55, 2 h00, 2 h05, 2 h10, 2 h15, 2 h20, 2 h25, 2 h30, 2 h35, 2 h40, 2 h45, 2 h50, 2 h55 and 3 h00.

In certain embodiments, the yield of the method is at least about 25%, preferably at least about 50%, preferably at least about 75%, more preferably at least about 90%. As used herein, the term “yield” is intended to refer to the amount of circularized nucleic acid molecules over the amount of total nucleic acid molecules (both linear and circular nucleic acid molecules). Within the scope of the invention, the expression “at least about 25%” includes at least about 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 81%, 82%, 83%, 84%, 85%, 86%, 87%, 88%, 89%, 90%, 91%, 92%, 93%, 94%, 95%, 96%, 97%, 98%, 99% and 100%.

In some embodiments, the yield of circularization is about 95%.

Another aspect of the invention relates to a circularized double stranded DNA nucleic acid obtainable by a method according to the instant invention.

In some embodiments, the circularized double stranded DNA nucleic acid is functionalized and/or relaxed.

In certain embodiments, the circularized double stranded DNA nucleic acid is a functionalized DNA nucleic acid. In certain embodiments, the circularized double stranded DNA nucleic acid is a relaxed DNA nucleic acid. In certain embodiments, the circularized double stranded DNA nucleic acid is a functionalized and relaxed DNA nucleic acid.

In some embodiments, the circularized double stranded DNA nucleic acid is a supercoiled DNA nucleic acid. The topology of circularized DNA produced with the use of the instant invention may be further modified with proteins well known in the state of the art, for example such as topoisomerases of types I, II, III, IV, V and VI.

One aspect of the invention pertains to a host cell comprising a circularized double stranded DNA nucleic acid obtainable by a method according to the instant invention.

In some embodiments, the host cell according to the invention may be a eukaryotic cell or a prokaryotic cell.

Non limitative examples of eukaryotic host cells may include animal cells, fungi cells, plant cells or yeast cells. In certain embodiments, the host cell according to the invention may be an animal cell, including e.g., a non-human mammal cell and a human cell.

In certain embodiments, an animal host cell according to the instant invention may be selected in a non-limitative group comprising a cell of the central nervous system, an embryonic cell, an endothelial cell, an epithelial cell, a germ cell, a hematopoietic progenitor cell, a hematopoietic stem cell, an induced Pluripotent Stem Cell (iPSC), a muscular cell, a progenitor cell, a stem cell, and the like.

In practice, animal ESCs, in particular human ESCs may advantageously be obtained without embryo destruction, as described by Chung et al. (2008), or by parthenogenetic activation of an unfertilized oocyte, as described by Sagi et al. (2016).

In some embodiments, the host cell may belong to a tissue selected in a non-limitative group comprising a connective tissue, an endothelial tissue, an epithelial tissue, a muscle tissue, a nervous tissue, a vascular tissue, and the like.

In some embodiments, the host cell may belong to an organ selected in a non-limitative group comprising a bladder, a bone, a brain, a breast, a central nervous system, a cervix, a colon, an endometrium, a kidney, a larynx, a liver, a lung, an esophagus, an ovarian, a pancreas, a pleura, a prostate, a rectum, a retina, a salivary gland, a skin, a small intestine, a soft tissue, a stomach, a testis, a thyroid, an uterus, a vagina.

In some embodiments, the host cell may non-limitatively originate from a human or a non-human animal, in particular a dog, a cat, a mouse, a rat, a fly, a rabbit, a pig, a chicken, a mosquito, a zebrafish, a horse and a cow.

In certain embodiments the host cell may originate from a vegetal in particular, bean, corn, rice, soy, tomato, and wheat.

In some embodiments, the host cell may be a microorganism, in particular selected in a group comprising archaea, bacteria and protozoa.

In some embodiments, suitable bacteria may be exclusively non-pathogenic bacteria. As used herein, the term “non-pathogenic” refers to a bacterium that does not harm or cause an infection in a target vegetal or animal, in particular a mammal animal, more preferably a human. In some alternative embodiments, suitable bacteria may be an attenuated pathogenic bacterium. As used herein, the expression “attenuated pathogenic” refers to a bacterium that has a reduced virulence, as compared to a non-attenuated pathogen. Non-limitative examples of attenuated pathogenic bacteria may comprise attenuated bacteria of the genus Pseudomonas, preferably P. aeruginosa; of the genus Listeria, preferably L. monocytogenes.

In some embodiments, the bacterium is a Gram-negative bacterium. In practice, the Gram coloration may be performed according to the methods well described in the state of the art. In some embodiments, the bacterium is from the family of Enterobacteriaceae. In some embodiments, said bacterium is of the genus Escherichia, preferably of the species E. coli.

In some embodiments, the bacterium is a Gram-positive bacterium. Non-limitative examples of Gram-positive bacteria include bacteria of the genus Bifidobacterium, and lactic acid bacteria (LAB), in particular of the genus Abiotrophia, Aerococcus, Carnobacterium, Enterococcus, Lactobacillus, Lactococcus, Leuconostoc, Oenococcus, Pediococcus, Streptococcus, Tetragenococcus, Vagococcus and Weissella.

In some embodiments, the host cell may be a genetically modified cell.

Circularized double stranded DNA nucleic acids according to the invention may be incorporated in the host cell by any suitable method known in the state of the art, or a method adapted therefrom. In practice, bacterial host cells may be transformed, whereas the eukaryotic host cells may be transfected with the circularized double stranded DNA nucleic acids according to the invention. Chemical transformation or transfection methods may encompass the use of calcium chloride. Mechanical transformation or transfection methods may include electroporation. For detailed protocols for transforming or transfecting host cells, one may refer to Sambrook et al. (Molecular Cloning—A Laboratory Manual—2^{nd} Edition; Cold Spring Harbor Laboratory Press).

In some aspect, the circularized double stranded DNA nucleic acid according to the instant invention is for use as a medicament.

The invention further relates to the use of a circularized double stranded DNA nucleic acid according to the instant invention for the preparation or the manufacture of a medicament.

A still other aspect of the invention relates to the circularized double stranded DNA nucleic acid according to the invention for use in gene therapy, and/or in DNA vaccination, and/or in cell therapy, and/or in genome editing, and/or in the production of induced pluripotent stem cells, and/or in the transfection or transformation of cultured cells. Another aspect of the invention relates to the use of the circularized double stranded DNA nucleic acid according to the invention for gene therapy, and/or for DNA vaccination, and/or for cell therapy, and/or for genome editing, and/or for the production of induced pluripotent stem cells, and/or for the transfection or transformation of cultured cells.

A further aspect of the invention relates to a method for implementing gene therapy, and/or DNA vaccination, and/or cell therapy, and/or genome editing, and/or production of induced pluripotent stem cells, and/or transfection or transformation of cultured cells, comprising at least step a) to step g), as defined herein.

As used herein, the expression “gene therapy” is intended to refer to a therapeutic technique that relies upon genes to treat or prevent a disease. In practice, this technique consists in introducing a gene into a patient's cells, in order to either replace a mutated gene that causes disease with a healthy copy of the gene, inactivate (or “knock out”) a mutated gene that is functioning improperly, or introduce a new gene into the body to help fight a disease.

As used herein, the expression “DNA vaccination” is intended to refer to the direct introduction into appropriate tissues of a DNA vector containing the DNA sequence encoding the antigen(s) against which an immune response is sought, and relies on the in-situ production of the target antigen (World Health Organization).

A used herein, the expression “cell therapy” is intended to refer to a therapeutic technique that relies upon cells to treat or prevent a disease. In practice, this technique consists in transplanting cells into a diseased tissue or organ in order to restore the tissue or organ function.

In some embodiments, the cell therapy includes therapy mediated by adoptive T cell therapy, in particular chimeric antigen receptor (CAR) T-cells. There are several forms of adoptive T cell therapy being used for cancer treatment: culturing and expanding tumor infiltrating lymphocytes (TIL), isolating and expanding one particular lymphocyte T cell clone, as well as using T cells that have been engineered to express a chimeric antigen receptor (CAR) to potently recognize tumor antigens and destroy tumor cells. In certain embodiments, the circularized DNA nucleic acids according to the invention encode a chimeric antigen receptor (CAR).

As used herein, the expression “genome editing” is intended to refer to the targeted insertion, deletion, modification or substitution of nucleic acids within the recipient's genome.

The invention also relates to the therapeutic use of a circularized double stranded DNA nucleic acid according to the invention for preventing and/or treating a disorder.

The invention further relates to a method for preventing and/or treating a disorder in an individual in need thereof, comprising the administration of a therapeutic effective amount of a circularized double stranded DNA nucleic acid according to the invention.

In some embodiments, the disorder may be selected in a non-limitative group comprising a cancer, a genetic disorder, an infectious disease and a neurodegenerative disease.

In some embodiments, the cancer is selected in a non-limitative group comprising a bladder cancer, a bone cancer, a brain cancer, a breast cancer, a central nervous system cancer, a cervix cancer, a cancer of the upper aero digestive tract, a colorectal cancer, an endometrial cancer, a germ cell cancer, a glioblastoma, a Hodgkin lymphoma, a kidney cancer, a laryngeal cancer, a leukemia, a liver cancer, a lung cancer, a myeloma, a nephroblastoma (Wilms tumor), a neuroblastoma, a non-Hodgkin lymphoma, an esophageal cancer, an osteosarcoma, an ovarian cancer, a pancreatic cancer, a pleural cancer, a prostate cancer, a retinoblastoma, a skin cancer, a small intestine cancer, a soft tissue sarcoma, a stomach cancer, a testicular cancer and a thyroid cancer.

In some embodiments, the genetic disorder may be selected in the non-limitative group comprising Achondroplasia, Alpha-1 Antitrypsin Deficiency, Antiphospholipid Syndrome, Autism, Autosomal Dominant Polycystic Kidney Disease, Breast cancer, Charcot-Marie-Tooth, Colon cancer, Cri du chat, Crohn's Disease, Cystic fibrosis, Dercum Disease, Down Syndrome, Duane Syndrome, Duchenne Muscular Dystrophy, Fanconi Anemia, Factor V Leiden Thrombophilia, Familial Hypercholesterolemia, Familial Mediterranean Fever, Fragile X Syndrome, Gaucher Disease, Hemochromatosis, Hartnup's Disease, Hemophilia, Holoprosencephaly, Huntington's disease, Kartagener's Syndrome, Klinefelter syndrome, Marfan syndrome, Myotonic Dystrophy, Neurofibromatosis, Noonan Syndrome, Osteogenesis Imperfecta, Parkinson's disease, Phenylketonuria, Poland Anomaly, Porphyria, Progeria, Prostate Cancer, Retinitis Pigmentosa, Severe Combined Immunodeficiency (SCID), Sickle cell disease, Skin Cancer, Spinal Muscular Atrophy, Tay-Sachs, Thalassemia, Trimethylaminuria, Tuberous Sclerosis, Turner Syndrome, Velocardiofacial Syndrome, WAGR Syndrome and Wilson Disease.

In some embodiments, the infectious disease may be selected in the non-limitative group comprising Acute rheumatic fever, Anthrax, Australian bat lyssavirus, Avian influenza (Bird Flu), Babesiosis, Barmah Forest virus, Botulism, Brucellosis, Campylobacteriosis, Chancroid, Chickenpox, Chikungunya, Chlamydia, Cholera, Creutzfeldt-Jakob disease (CJD), Cryptosporidiosis, Cytomegalovirus (CMV), Dengue, Dientamoeba fragilis, Diphtheria, Donovanosis, Ebola virus disease, Epidemic keratoconjunctivitis, Epstein-Barr virus (EBV), Fifth disease, Gastroenteritis, German measle (Rubella), Giardiasis, Gonorrhoea, Glandular fever (Infectious mononucleosis), Haemolytic uraemic syndrome, Haemophilus influenzae Type b (Hib), Hand foot and mouth disease, Hendra virus, A/B/C/D/E Hepatitis, Human immunodeficiency virus (HIV), Influenza, Japanese encephalitis, Kunjin virus, Legionnaires' disease, Leprosy, Leptospirosis, Listeriosis, Lyme disease, Lymphogranuloma venereum (LGV), Malaria, Maternal sepsis (Puerperal fever), Measles, Meningococcal disease, MERS coronavirus, MRSA, Mumps, Murray Valley encephalitis (MVE), Norovirus, Pandemic influenza, Parvovirus B19, Pertussis, Plague, Pneumococcal disease, Poliomyelitis, Psittacosis, Q fever, Rabies, Rat Lung worm, Respiratory syncytial virus (RSV), Rheumatic heart disease, Rickettsia, Ross River virus, Rotavirus, Rubella, Salmonellosis, SARS coronavirus, Shiga toxigenic E. Coli (STEC/VTEC), Shigellosis, Shingles, Smallpox, Syphilis, Tetanus (lock-jaw), Tuberculosis (TB), Tularemia, Typhoid, Typhus, Varicella-Zoster virus, Viral haemorrhagic fevers, Whooping cough, Yellow fever and Zika virus.

In some embodiments, the neurodegenerative disease may be selected in the non-limitative group comprising Alzheimer's disease, Amyotrophic lateral sclerosis, Down's syndrome, Friedreich's ataxia, Huntington's disease, Lewy body disease, Parkinson's disease and Spinal muscular atrophy.

In some aspects, the invention relates to the use of the circularized double stranded DNA nucleic acid according to the instant invention, for the storage of data, and/or for the sequencing of nucleic acids, and/or for the production of rolling circle DNA, and/or for the production of proteins, and/or for the production of RNA, and/or in metabolic pathway engineering, and/or in molecular biology, and/or for the transformation of bacteria, and/or for the production of viruses.

A further aspect of the invention relates to a method for implementing the storage of data, and/or the sequencing of nucleic acids, and/or the production of rolling circle DNA, and/or the production of polypeptides, and/or the production of RNA, and/or metabolic pathway engineering, and/or molecular biology, and/or the transformation of bacteria, and/or for production of viruses comprising at least step a) to step g), as defined herein.

In some aspects, the invention also relates to a method for implementing the storage of data, and/or the sequencing of nucleic acids, and/or the production of rolling circle DNA, and/or the production of polypeptides, and/or the production of RNA, and/or metabolic pathway engineering, and/or molecular biology, and/or the transformation of bacteria, and/or for production of viruses comprising at least a step of implementing a circularized nucleic acid obtained by a method according to the invention.

In some embodiments, the circularized nucleic acid according to the invention may encode one or more polypeptide(s), or one or more RNA(s). In said embodiments, the circularized nucleic acid according to the invention may be suitable for the production of polypeptides, or for the production of RNA. Transfection of eukaryotic cells or transformation of bacteria may be performed with the circularized nucleic acid according to the invention, by the mean of any suitable method known from the state of the art, or a method adapted therefrom. In one embodiment, the circularized nucleic acid according to the invention may encode one or more polypeptide involved in the metabolic pathway. In said embodiment, the circularized nucleic acid according to the invention may be useful to reconstitute a metabolic pathway into the host cell.

In certain embodiments, the circularized nucleic acid according to the invention may be used as a vector in order to clone nucleic acids of interest in microorganisms or to propagate the vector through replication in eukaryotic cells. In other words, the circularized nucleic acid according to the invention may provide a tool for molecular biology.

In some embodiments, the circularized nucleic acid according to the invention may encode the genetic material for producing viruses.

In practice, for the storage of data, digital data may be stored in the form of the circularized nucleic acid according to the invention. In said embodiments, the NHR1 or NHR2 nucleic acids may serve as index, i.e. may represent information suitable for easily retrieving the digital data from the vial containing a set of different digital data stored in the form of circularized DNA molecules. The digital data may be deciphered with the use of a DNA sequencer machine, preferentially based on those technologies that require DNA molecules providing the features of the rolling circle DNA molecules, preferentially the technology of Pacific Biosciences®.

In another embodiment, the circularized nucleic acids obtained with the use of the instant invention may be applied for all those applications when the rolling circle DNA molecules are needed.

In another aspect, the invention relates to a kit for the circularization of a double stranded DNA nucleic acid, said kit comprising:
a) a polypeptide having a 3′-5′ nuclease activity;
b) a polypeptide having a 5′-3′ nuclease activity;
c) a DNA polymerase;
d) a mesophilic DNA ligase; and optionally,
e) one or more buffer(s).

In some embodiments, the polypeptide having a 5′-3′ nuclease activity, the DNA polymerase and the DNA ligase are in the form of a mixture.

In some embodiments, said kit further comprises one or more buffer(s).

In certain embodiments, the kit comprises a buffer comprising ATP and dNTPs.

In some embodiments, the kit comprises at least two vials:
the first vial comprising the polypeptide having a 3′-5′ nuclease activity; and optionally alkaline phosphatase and/or a polypeptide with a type I exonuclease activity;
the second vial comprising a DNA ligase; and optionally a polypeptide having a 5′-3′ nuclease activity, and/or a DNA polymerase with no strand displacement activity and with no 5′ to 3′ exonuclease activity, ATP and dNTPs.

In some embodiments, the kit comprises at least two vials:
the first vial comprising the polypeptide having a 3′-5′ nuclease activity;
the second vial comprising a DNA ligase.

In some embodiments, the first vial further comprises alkaline phosphatase and/or a polypeptide with a type I exonuclease activity. In certain embodiments, the second vial further comprises a polypeptide having a 5′-3′ nuclease activity. In some embodiments, the second vial further comprises a DNA polymerase with no strand displacement activity and with no 5′ to 3′ exonuclease activity, and dNTPs.

In certain embodiments, the polypeptide having a 3′-5′ nuclease activity and optionally alkaline phosphatase and/or the polypeptide with a type I exonuclease activity within the first vial are in an appropriate buffer. In some embodiments, DNA ligase, and optionally the polypeptide having a 5′-3′ nuclease activity, the DNA polymerase with no strand displacement activity and with no 5′ to 3′ exonuclease activity and dNTPs within the second vial are in an appropriate buffer.

In alternative embodiments, appropriate buffers are comprised in separate vials.

In some embodiments, the kit further comprises one or more primers and/or oligonucleotides with functional modifications.

In certain embodiments, the kit further comprises one or more primer(s) selected in the group consisting of a primer of sequence SEQ ID NO. 43 (TTAGATTAGTTCATGGTCATAGCTGTTTCCTGTTTTCCCAGTCACGACG) and a primer of sequence SEQ ID NO. 44 (CCTGAGCGGATAACAATTTCACAC).

Advantages of the method disclosed herein allowing the production of circularized double stranded nucleic acids, are developed hereunder.

As illustrated in the examples below, the above-described method allows producing circularized nucleic acid molecules:
in a fast, cheap and simple manner, as compared to existing methods;
in large quantities as compared to existing methods (increased yields);
from PCR products or existing vectors (comprising for example a bacterial and/or viral backbone)
that may be 100% of human origin (bacteria and/or viral DNA-free), and hence that comply with safety issue when applied to human medicine;
that may have longer persistence in cells;
that may possess higher transfection efficiency (up to 2 or 4 times), as compared to existing methods;
that may promote longer gene expression over the time;
that may be fitted to regulations;
that may behave as an extra-chromosome, when comprising a S/MAR nucleic acid sequence;
that may be compatible with existing technologies such as, e.g., lentivirus, sleeping beauty, CRISPR/Cas9, “DNA minicircles”, and replicative episome;
that may further be functionalized easily in vitro, therefore avoiding the necessary presence of living organisms.

### BRIEF DESCRIPTION OF THE DRAWINGS

FIG. 1 is a scheme illustrating one embodiment of the invention. The starting dsDNA nucleic acid comprises TBC, HR2, NHR2. Following a PCR step, with suitable primers, an amplicon of formula NHR1-HR1-TBC-HR2-NHR2 is obtained. Said amplicon is incubated with Exo III nuclease in order to generate 5′-overhangs. An annealing step generate a circular DNA nucleic acid comprising 2 gaps and 2 non-hybridized 5′-overhangs. The 2 gaps are then filled in the presence of a DNA polymerase and the 5′-overhangs are further removed in the presence of nuclease with 5′-3′ activity (Exo 5′-3′), hereby generating a circular dsDNA comprising 2 nicks. These 2 nicks are finally sealed in the presence of a ligase. The final product is circularized dsDNA nucleic acid, in which the 2 strands are continuous.

FIG. 2 is a scheme illustrating one embodiment of the invention. The starting dsDNA nucleic acid is plasmid comprising a nucleic acid of formula NHR1-SOR1-HR1-TBC-HR2-SOR2-NHR2. The starting dsDNA nucleic acid is digested in the presence of restriction enzymes cleaving the corresponding restriction sites on SOR1 and SOR2, and in the presence of nicking restriction enzymes, that cleave the nick restriction sites on each strand of TBC. One nick restriction site is localized at distance of the 3′ end of HR1, and the other nick restriction site is localized at 0 nucleotide of the 3′ end of HR2. The resulting nucleic acid comprises 2 nicks and 2 3′-overhangs, which cannot be digested by the Exo III nuclease. Digestion by Exo III nuclease generates a dsDNA nucleic acid with 3′-overhangs, since digestion initiated at the nick restriction sites. Annealing generates a circular dsDNA nucleic acid with 1 gap and 1 nick. The gap is filled by the mean of an oligonucleotide, which results in a dsDNA nucleic acid having 3 nicks, which are sealed in the presence of a ligase.

FIG. 3 is a scheme illustrating one embodiment of the invention. The starting dsDNA nucleic acid comprises TBC, HR2, NHR2′, wherein NHR2′ represent partial NHR2. Following a PCR step, with suitable primers, an amplicon of formula NHR1-HR1-TBC-HR2-NHR2 is obtained. NHR1 further comprises a restriction site. Said amplicon is incubated with the restriction enzyme that cleaves the restriction site within NHR1, which generates a dsDNA nucleic acid of formula HR1-TBC-HR2-NHR2, in which the HR1 presents a 5′-overhang. The resulting dsDNA nucleic acid is digested in the presence of an Exo III nuclease in order to generate 5′-overhangs. An annealing step generates a circular dsDNA nucleic acid comprising 2 gaps and 1 non-hybridized 5′-overhangs, formed of NHR2. The 2 gaps are filled in the presence of a DNA polymerase, generating 2 nicks. In said embodiment, the 5′-overhangs is not removed, and a ligase seals only one of the 2 nicks, generating a circular dsDNA nucleic acid, in which only one of two strands is continuous, also named “rolling circle DNA”. The NHR2 5′-overhang may be useful as an index, e.g., when the rolling circle DNA is intended to be employed for data storage.

FIG. 4 is a photograph showing the efficacy of intramolecular annealing of dsDNA with respect to the annealing temperature. Lane L: ladder (the 1.5 kbp, 5.0 kbp and 20.5 kbp nucleic acids are represented on the left side); lane 1: 80° C.; lane 2: 75° C.; lane 3: 70° C.; lane 4: 65° C.; lane 5: 60° C.; lane 6: 55° C.; lane 7: 50° C. Arrows indicate the nature of the nucleic acid; a: intramolecularly annealed dsDNA; b: trimers; c: dimers; d: linear DNA (3.8 kbp).

FIG. 5 is a photograph showing that the treatment of non-purified PCR product with Exo III, rSAP and Exo I improves the output of intramolecular annealing of dsDNA. Lane L: ladder; lane K: control, amplicons DNA before treatments and without purification (linear; 3.8 kbp); lane A: 12 min treatment on ice with mix of 5u Exo I+1u rSAP+25u Exo III in 50 μL of final 0.5×MgAcetate containing 5 μL of PCR product; lane B: 3 min treatment on ice with mix of 5u ExoI+1u rSAP+25u Exo III in 50 μL of final 0.5×MgAcetate containing 5 μL of PCR product; lane 1: only 5u Exo I treatment at 37° C. for 20 min and 25u Exo III on ice for 3 min or 10 min; lane 2: 1 u rSAP treatment at 37° C. during 20 min and 25u Exo III on ice during 3 min or 10 min; lane 3: simultaneous treatment with 1u rSAP and 5u Exo I at 37° C. for 20 min and 25u Exo III on ice during 3 min or 10 mM; lane 4: 25 u Exo III on ice during 3 mM or 10 mM (no rSAP and no Exo I treatments). Arrows indicate the nature of the nucleic acid; a: intramolecularly annealed dsDNA; b: dimers; c: linear DNA (3.8 kbp).

FIG. 6 is a photograph showing the stability of intramolecularly annealed dsDNA with respect to the ratio DNA/Exo III. Lane L: ladder; lane K: control, amplicons DNA before treatments; lane 1: 100 ng/10 μL; lane 2: 300 ng/10 μL; lane 3: 700 ng/10 μL; lane 4: 900 ng/10 μL. Amounts of Exo III are indicated in the upper part of the photograph (10u, 20u and 40u). Arrows indicate the nature of the nucleic acid; a: intramolecularly annealed dsDNA; b: dimers; c: linear DNA (3.8 kbp).

FIG. 7 is a photograph showing the efficiency of intramolecular annealing of dsDNA in the presence of Exo I. Lane L: ladder; lane 1: 10 u Exo III; lane 2: 40 u Exo III. Arrows indicate the nature of the nucleic acid; a: intramolecularly annealed dimeric dsDNA; b: intramolecularly annealed monomeric dsDNA; c: linear DNA (3.8 kbp).

FIG. 8 is a photograph showing the efficiency of intramolecular annealing of dsDNA with respect to the amount of T4 DNA Polymerase. Lane 1: purified amplicon 1.8 kbp 110 ng/10 μL; lane 2: purified amplicon 3.8 kbp 140 ng/10 μL; lane 3: non-purified PCR product with amplicon 3.8 kbp 2 μL in 10 μL of reaction (Exo III+Exo I+rSAP). No treatment. indicates the absence of any treatment. Temperature of treatment is indicated at the upper part of the photograph. Arrows indicate the nature of the nucleic acid; a: intramolecularly annealed dsDNA; b: dimers; c: linear DNA (3.8 kbp); d: dimers; e: dimers with filled gaps; f: linear DNA (1.8 kbp).

FIG. 9 is a photograph showing the efficiency of circularization with respect to the ratio between DNA ligase, Exo VII and T4 DNA Polymerase. The buffer contains 2 mM ATP and 2 mM dNTP. Lane A: Amplified fragment before circularization; lane B: After Exo III 100u for 5 mM at RT° of 3000 ng of DNA in 100 μL 0.5×MgAcetate buffer and anneal 88° C. for 5 min, 50° C. for 15 min; lane 1: 40 u DNA Ligase, 1u Exo VII and 5u T4 Pol; lane 2: 40 u DNA Ligase, 0.1u Exo VII and 5u T4 Pol; lane 3: 40 u DNA Ligase, 0.01u Exo VII and 5u T4 Pol; lane 4: 40 u DNA Ligase, 1u Exo VII and 0.5u T4 Pol; lane 5: 40 u DNA Ligase, 0.1u Exo VII and 0.5u T4 Pol; lane 6: 40 u DNA Ligase, 0.01u Exo VII and 0.5u T4 Pol; lanes 1-6: 300 ng of DNA in 20 μL. Arrows indicate the nature of the nucleic acid; a: intramolecularly annealed dsDNA; b: dimers; c: linear DNA (3.8 kbp).

FIG. 10 is showing the efficiency of circularization with respect to the ratio between DNA ligase, Exo VII and T4 DNA Polymerase. The buffer contains 2 mM ATP and 2 mM dNTP. Lane A: Amplified fragment before circularization; lane B: After Exo III 100u for 5 mM at RT° of 3000 ng of DNA in 100 μL 0.5×MgAcetate buffer and anneal 88° C. for 5 min, 50° C. for 15 min; lane 1: 200 u DNA Ligase, 5u Exo VII and 2.5u T4 Pol; lane 2: 100 u DNA Ligase, 2.5u Exo VII and 1.25u T4 Pol; lane 3: 50 u DNA Ligase, 1.25u Exo VII and 0.6u T4 Pol; lane 4: 25 u DNA Ligase, 0.6u Exo VII and 0.3u T4 Pol; lane 5: 12 u DNA Ligase, 0.3u Exo VII and 0.15u T4 Pol; lane 6: 6 u DNA Ligase, 0.15u Exo VII and 0.07u T4 Pol; lane 7: 3 u DNA Ligase, 0.07u Exo VII and 0.04u T4 Pol; lane 8: 1.5u DNA Ligase, 0.04u Exo VII and 0.02u T4 Pol; lanes 1-8: 300 ng of DNA in 20 μL. Arrows indicate the nature of the nucleic acid; a: intramolecularly annealed dsDNA; b: dimers; c: linear DNA (3.8 kbp).

FIG. 11 is a scheme illustrating one embodiment of the invention. 5′ phosphates ends and thiophosphate nucleotide analogues may be introduced in the HR1 and HR2 and circularization may be obtained via a two-step process.

FIG. 12 is a scheme illustrating one embodiment of the invention in the presence of thiophosphate nucleotide analogues in the linear double stranded nucleic acid to be circularized.

FIG. 13 is a scheme illustrating circularization process of FIG. 13, in which the step of recessing the ends of the linear double stranded nucleic acid to be circularized by Lambda exonuclease, ExoI et ExoIII are further detailed.

FIG. 14 is a photograph showing the efficiency of DNA molecules circularization as being depend on magnesium concentration. The DNA molecules were incubated with Exonuclease III in a buffer containing different magnesium acetate (MgAc) concentration (from 0 mM to 26 mM) for 2 mM at 30° C., 10 mM at 75° C., 5 mM at 60° C. and 1 mM 4° C., successively. Lane 1: molecular weight marker (see corresponding indication on a side in bp); lane 2: 0 mM MgAc; lane 3: 2 mM MgAc; lane 4: 6 mM MgAc; lane 5: 10 mM MgAc; lane 6: 14 mM MgAc; lane 7: 18 mM MgAc; lane 8: 22 mM MgAc; lane 9: 26 mM MgAc; lane 10: molecular weight marker is relaxed circular DNA of a plasmid 3868 bp obtained by treatment with a nickase Nt.BbvCI; lane 11: molecular weight marker (in bp). Arrows indicate the nature of the nucleic acid; a: circular 4,528 bp nucleic acid; b: concatemers of the 4,528 bp fragments; c: linear 4,528 bp fragment; d: linear 1,901 bp (from the plasmid backbone).

FIG. 15 is a photograph showing that the method according to the invention provides high yields of circularized DNA molecules at high concentration of DNA molecules. The DNA molecules were incubated with Exonuclease III in 30 mM magnesium acetate buffer for 2 mM at 30° C., 10 mM at 75° C., 5 mM at 60° C. and 1 min at 4° C., successively. 30 ng/μL of DNA molecules in 12 μL reaction volume were treated with 20u of Exonuclease III (lane 3) and 150 ng/μL DNA molecules in 50 μL were treated with 100u of Exonuclease III (lane 4). Lane 1: molecular weight markers (bp; see corresponding indication on a side); lane 2: relaxed circular DNA of a plasmid 3,868 bp obtained by treatment with a nickase Nt.BbvCI; lane 3: DNA molecules treated at 30 ng/μL concentration; lane 4: DNA molecules treated at 150 ng/μL concentration; lane 5: molecular weight marker. Arrows indicate the nature of the nucleic acid; a: circularized 4,528 bp DNA molecule; b: linear 1,901 bp fragment (from the plasmid backbone).

FIG. 16 is a set of photographs showing that the DNA molecules produced with the circularization method according to the invention promote an increased efficiency of cell transfection. FIG. 16A: Human osteosarcoma cells (HOS ATCC® CRL-1543™) transfected with circular 4,528 bp DNA molecules (black bars) display higher percentage of GFP-positive cells compared to parental 6,711 bp plasmid DNA (white bars) at all tested transfection conditions. The transfection conditions were 1,000 ng, 750 ng, 500 ng or 250 ng of DNA molecules applied for transfection per well in 24-well plate. FIG. 16B: Lung carcinoma cells of A549 cell line (ATCC® CCL185™) transfected with circular 4,528 bp DNA molecules (black bars) display higher percentage of GFP-positive cells compared to parental 6,711 bp plasmid DNA (white bars) in all tested transfection conditions.

### EXAMPLES

The present invention and disclosure are further illustrated by the following examples.

### Example 1: Embodiments According to the Invention

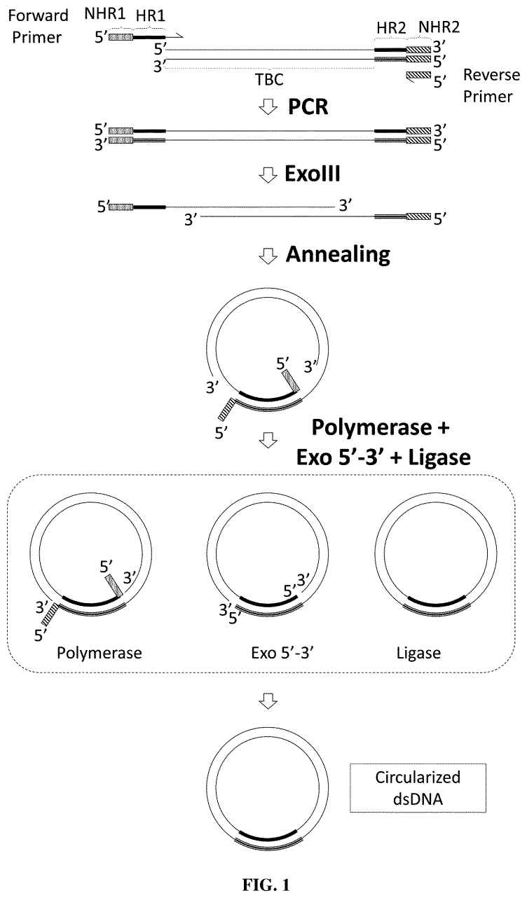

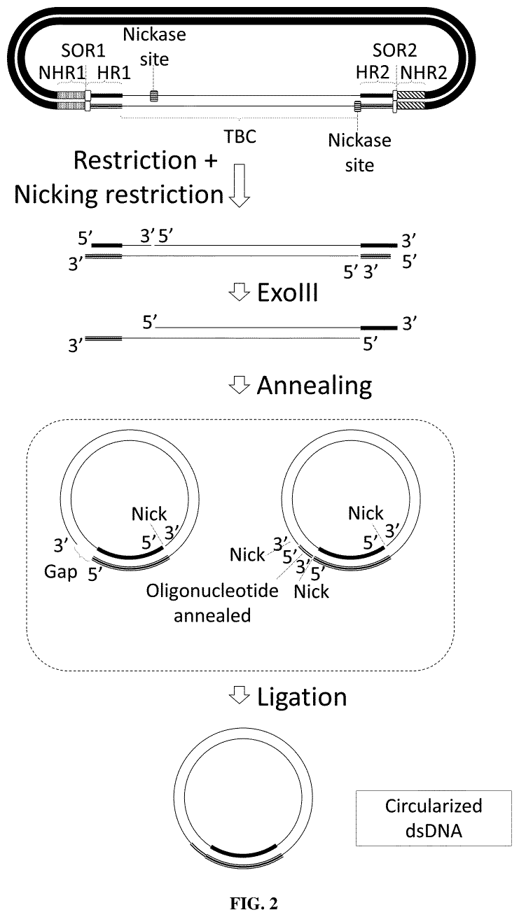

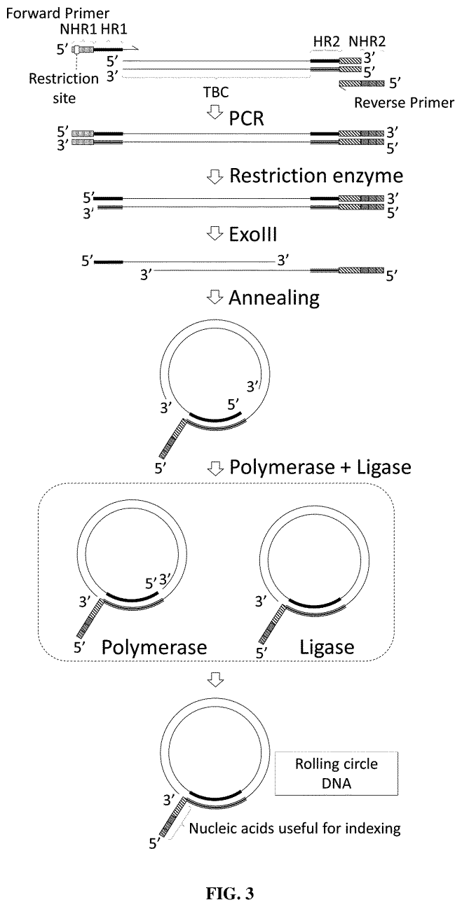

FIGS. 1, 2 and 3 illustrate three embodiments for obtaining a circularized dsDNA nucleic acid according to the invention (see the corresponding legends).

### Example 2: Protocol for Circularizing a DNA Nucleic Acid

1) Use one PCR tube to treat 1 μg to 9 μg of purified amplicons or 25 μL of raw PCR product in final 100 μL of 0.5×MgAcetate buffer with 10u to 200u of Exo III at 0° C. to 4° C. during 5 to 10 minutes. Optionally, treat simultaneously with 1u to 20u of Exo I in order to eliminate unused primers and with 1u to 10u rSAP in order to eliminate unused dNTP. The thermal treatments are preferentially performed in the PCR machine. The result of this step is the mild degradation of 3′-ends of double stranded molecules.

2) For denaturation, heat the tube at 88° C. during 5 minutes. Next for annealing, cool the tube to 45° C.-60° C. during at least 10 minutes (depending on melting temperature of the designed complementary sequence). The “universal” annealing step may be performed by gradual decrease of the temperature from 88° C. to 45° C. as following: 88° C. for 5 mM, 65° C. for 10 mM, 60° C. for 10 mM, 55° C. for 10 mM, 50° C. for 10 mM, 45° C. for 10 mM, successively. Complete the thermal treatments with cooling the tube to 4° C. The result of this step is the production of intramolecularly annealed dsDNA which contain single-stranded gaps and single-stranded overhangs. In some embodiments, this product may be directly applied for transformation of competent bacteria using an electroporation or thermal shock.

3) Add the mixture of enzymes (40u DNA Ligase, 1u Exonuclease VII and 5u T4 polymerase) diluted in a Buffer containing ATP (final concentration in reaction 1 mM-5 mM) and dNTP (final concentration in reaction 1 mM-5 mM) in order to repair and seal the gaps. Incubate at room temperature during 30 mM to 1 hour.

This product may be directly applied for transfection of cells in culture using a kit for transfection such as Lipofectamine® or for transformation of competent bacteria using an electroporation or thermal shock.

4) Add an exonuclease which will eliminate the molecules containing termini (preferentially EcoV/RecBCD but may be also Exonuclease VIII truncated or Lambda Exonuclease). Treat during 30 mM at 37° C. Stop the reaction with 25 mM EDTA and/or thermal treatment 70° C. for 30 mM This product may be directly applied for transfection of cells in culture using a kit for transfection such as Lipofectamine® or for transformation of competent bacteria using an electroporation or thermal shock.

5) To clean-up treated samples use one of the following steps: Column clean up or Running the reaction on an agarose gel, and then extracting the DNA or Performing a phenol/chloroform extraction followed by ethanol precipitation.

6) This product may be directly applied for transfection of cells in culture using a kit for transfection such as Lipofectamine® or for electroporation of cells in culture or for transformation of competent bacteria using an electroporation or thermal shock.

Unless specified otherwise, the buffer 0.5×MgAcetate Buffer (work dilution) is as follows:
25 mM Potassium Acetate (0-25 mM)
10 mM Tris-Acetate (10-50 mM)
5 mM Magnesium Acetate (5-15 mM)
50 μg/ml BSA (0-50 μg/ml)
pH 7.9 at 25° C. (pH 7.5-9.0).

This buffer is to be added at the step 1 of the protocol. This buffer preferentially contains ATP (final concentration in reaction 1 mM-5 mM) and dNTP (final concentration in reaction 1 mM-5 mM) if optional treatment with rSAP was void at the first step of the protocol.

A modified Mg/Acetate Buffer may be added at the step 3) of the protocol (Magnesium Acetate concentration may be at 0-1 mM and Potassium Acetate may be at 0-50 mM).

This buffer preferentially contains ATP (final concentration in reaction 1 mM-5 mM) and dNTP (final concentration in reaction 1 mM-5 mM) if ATP and dNTP were absent in the 0.5× Mg/Acetate Buffer to be added at the step 1 of the protocol.

### Example 3: Annealing Temperature Range

a) Experimental Design

Purified amplicons (3.8 kbp) 200 ng in 10 μL of 0.5×Acetate Buffer were treated with 10u Exo III during 3 min on ice. Annealing was performed as following: 88° C. for 5 mM, annealing temperature (80° C., 75° C., 70° C., 65° C., 60° C., 55° C. and 50° C.) for 3 mM, 3° C. for 10 sec. The melting temperature of the termini's complementary sequence (the sequence of M13 primer binding site) is 45° C.-50° C.

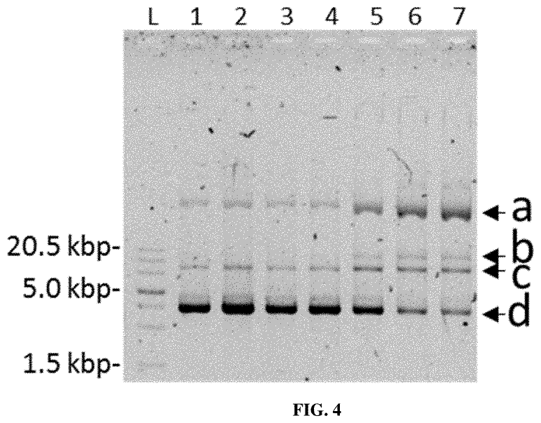

b) Results (FIG. 4)

FIG. 4 shows that intramolecularly annealed dsDNA (see arrow a) can be obtained at every annealing temperature tested. However, lower temperatures (60° C., 55° C. and 50° C.) result in a higher efficacy of intramolecular annealing (see the intensity of the intramolecularly annealed dsDNA (arrow a), as compared to the other forms (linear (arrow d); dimer (arrow c) and trimer (arrow b)).

### Example 4: ExoI and rSAP Treatment Improved Intramolecular Annealing Starting from Raw, Non-Purified PCR Product

a) Experimental Design

The experiments have been performed as follows: 5 μL of raw PCR product was diluted to 50 μL of 0.5×MgAcetate buffer. Enzymes Exo I and rSAP were added or not and incubated if necessary, at 37° C. during 20 mM. There was added 25u of Exo III, incubated for 3 mM or 10 mM or 12 mM and annealed using thermal treatment 88° C. for 5 min, 50° C. for 3 min, 4° C. for 1 min.

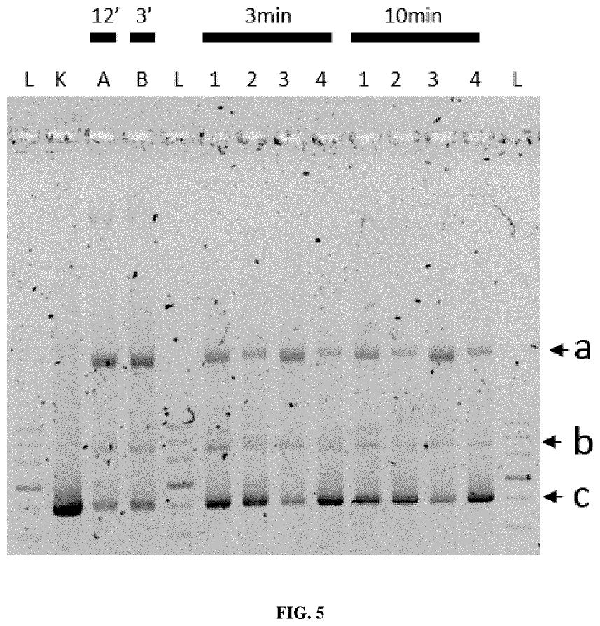

b) Results (FIG. 5)

The rSAP dephosphorylates dNTP nucleotides so that the polymerase from PCR reaction does not repair the degraded 3′-DNA strands. In addition, Exo I eliminates unused primers so that they do not compete with homologous sequences during circularization by annealing. The treatments by only Exo I or only rSAP during 20 min at 37° C. prior Exo III treatment and annealing display slight improvement or no improvement of intramolecular annealing as compared to the non-treated control DNA (see intense lower bands and relatively weak upper bands on Lanes 1, 2 and 4 in FIG. 5). The probes treated with rSAP, Exo I and Exo III show improvement of intramolecular annealing as compared to the non-treated control DNA (see weak lower bands and relatively intense upper bands on Lanes A, B and 3 in FIG. 5).

As a conclusion, non-purified PCR product can be intramolecularly annealed in the presence of a mix of Exo I, Exo III and rSAP.

### Example 5: Efficiency of Intramolecular Annealing with Respect to Amounts of Amplicon and Exo III

a) Experimental Design

100 ng to 900 ng of purified amplicons (3.8 kbp) were treated with different concentrations of Exo III (10u, 20u, 40u) in 10 μL of reaction volume and incubated during 5 minutes on ice. Annealing was performed at 88° C. for 5 min, 50° C. for 3 min, 3° C. for 1 min.

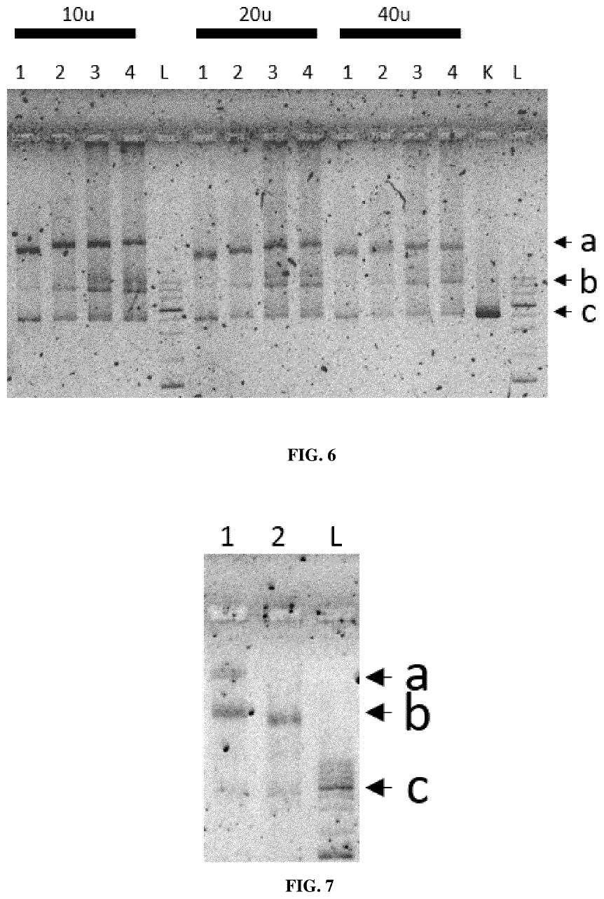

b) Results (FIG. 6)

FIG. 6 shows that the treatments at high Exo III concentrations lead to significant degradation of intramolecularly annealed dsDNA (compare the Lanes 1 to 4 after 10u, 20u and 40u of Exo III concentration (arrow a)). It was observed a correlation between the higher Exo III concentration and the weaker intensity of upper major band (arrow a; intramolecularly annealed dsDNA). Efficient intramolecular annealing was obtained for 100 ng and 300 ng amplicons after treatment with 10u Exo III (compare the intensity of the upper band and the central band on Lanes 1-2 to Lines 3-4 after 10u Exo III treatment in FIG. 6).

### Example 6: DNA Polymerase Range

a) Experimental Design

Purified amplicons (300 ng) were treated with Exo I during 30 min at 36° C. Next, the products were treated with 10u or 40u of Exo III during 10 min at room temperature (about 20° C.) in 10 μL of 0.5×MgAcetate Buffer. Thermal treatments were made as following: 88° C. for 5 min, 50° C. for 10 min.

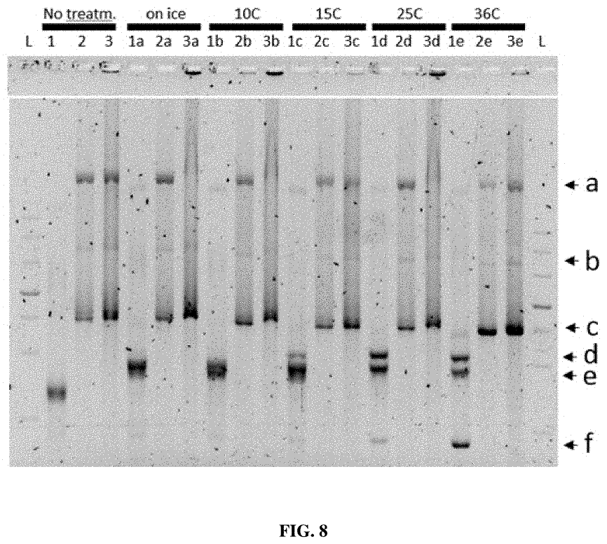

b) Results: (FIG. 7)

Almost all amplicons were intramolecularly annealed dsDNA after 10u and 40u treatments with Exo III (see only one major band on Lanes 1 and 2; see faint linear monomers and absence of linear dimers or trimers in FIG. 7).

### Example 7: Stability of the Intramolecularly Annealed dsDNA with Respect to the Temperature of DNA Elongation

a) Experimental Design

The probes labelled with letters are produced as follows: 5 μL of intramolecularly annealed dsDNA was mixed with 1 μL of mixture T4 DNA polymerase (5u/20 μL=0.25u/μL)

b) Results (FIG. 8)

T4 DNA polymerase has less potential “to open” intramolecularly annealed dsDNA molecules and “to monomerize” linear concatemers if applied at the temperature lower than 36° C. DNA monomers appear after elongation with T4 DNA polymerase at 36° C. (see low intense band on Lane 1e in FIG. 8). Optimal temperature for T4 polymerase-dependent elongation is at 25° C. or lower (compare the major band on Lane 1 to the major lower band on Lanes 1a, 1b, 1c and 1d in FIG. 8). The phenomena of “DNA monomerisation” derives from quasi-strand-displacement activity (that is absent in T4 polymerase) at relatively high temperature. DNA-wobbling “displaces” the complementary strand in front of T4 DNA polymerase, which progressively elongates the 3′-end through “displaced” strand until “opens” the circle of intramolecularly annealed dsDNA.

### Example 8: Efficiency of the DNA Circularization with Respect to the Reparation of Gaps

a) Experimental Design

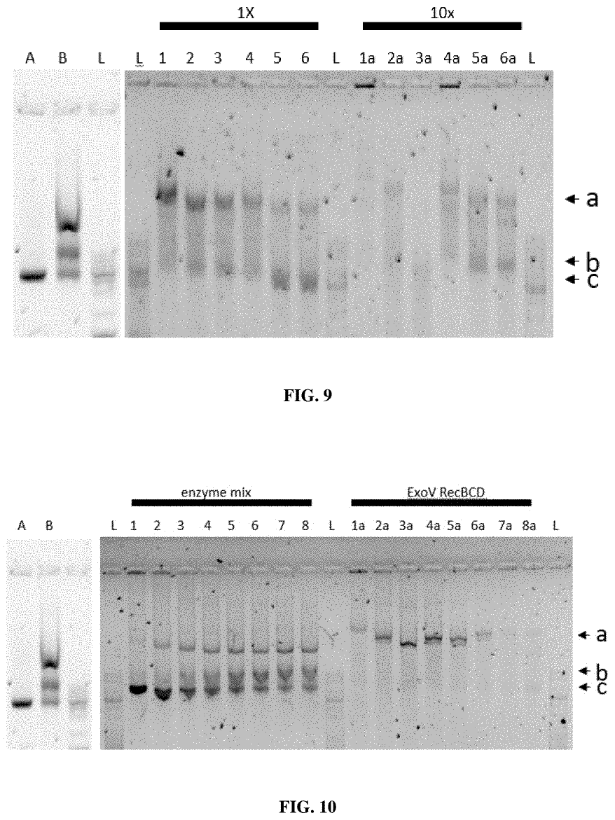

The intramolecularly annealed dsDNA molecules were first treated with different ratios (see below) of three enzymes in mixtures containing DNA Ligase, Exonuclease VII and T4 polymerase in 0.5×MgAcetate buffer with 1.25 mM dNTP, 0.5 mM ATP and 300 ng of DNA in 20 μL of reaction volume during 1 hour at room temperature (FIG. 9).

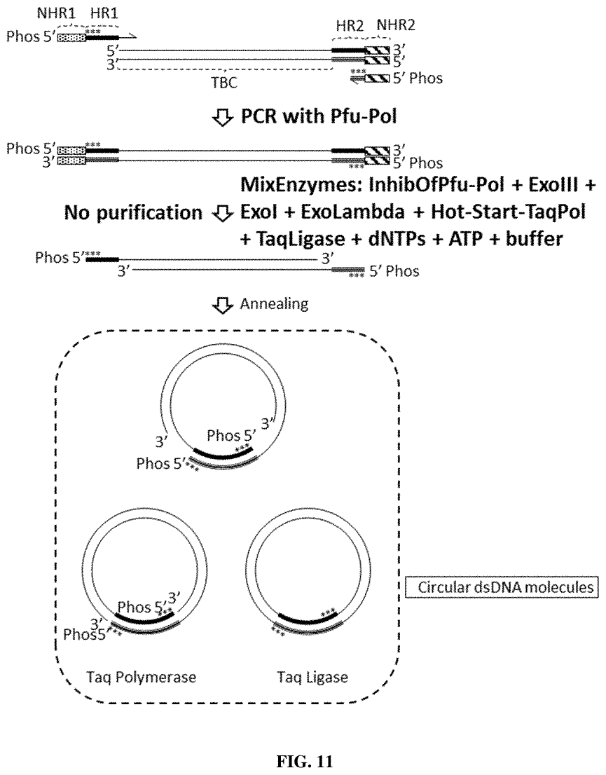

The intramolecularly annealed dsDNA molecules were further treated with 1 to 2 serial dilutions of enzyme mix containing DNA Ligase, Exonuclease VII and T4 polymerase in 0.5×MgAcetate buffer with 1.25 mM dNTP, 0.5 mM ATP and 300 ng of DNA in 20 μL of reaction volume during 1 hour at room temperature. Products were alternatively treated accordingly in the presence of ExoV/RecBCD (FIG. 10).

b) Results

The results depicted in FIG. 9 show that the 10× concentrations of mixed enzymes results in a decrease of circularization (see weak bands in 1a to 6a). In other words, the reaction of the polymerase, ExoVII and the ligase results in discontinuous strands in the circular dsDNA nucleic acid. The ratios of enzymes in mixtures are near to optimal in 1 to 4.

The results depicted in FIG. 10 show that:
the concentration of one or more than one of enzymes in the mix is above optimal in conditions of 1 and 2 that leads to transformation of intramolecularly annealed dsDNA and dimeric DNA into linear monomers (see intense lower band in 1 and 2 and relatively weak bands in 1a and 2a);
the concentration of enzymes is near to optimal in conditions of 3 and 4 because the production of circularized DNA is at maximum after Exo V/RecBCD treatment (see intense band in 3a and 4a) and because the production of linear monomers is moderate (see intense upper band and presence of dimers in 3 and 4);
the concentration of enzymes is lower than optimal in conditions of 5 to 8 because the intensity of circularized DNA band gradually decreases from 5 to 8 after Exo V/RecBCD treatment (see the band intensity in 5a, 6a, 7a and 8a).

### Example 9: Example of a Two-Step Circularization Protocol (FIGS. 11-13)

FIG. 11 shows that it is possible to introduce thiophosphate nucleotide analogs (asterisks), as well as 5′ phosphate ends (Phos 5′), in the HR1 and HR2 nucleic acids by the means of appropriate oligonucleotides (primers). Upon PCR amplification with the Pfu DNA polymerase, a linear double stranded nucleic acid is obtained, which can be circularized, without the purification step, in a one step process in the presence of an inhibitor of Pfu DNA polymerase (InhibOfPfu-Pol is an oligonucleotide containing uracil nucleotides), Exonuclease III (ExoIII), Exonuclease (ExoI), Exonuclease Lamdba (ExoLambda), Hot Start Taq DNA polymerase (Hot-Start-TaqPol), Taq DNA ligase (TaqLigase), dNTPS, ATP and the appropriate buffer. In practice, the 3′ ends are recessed in the presence of ExoIII and ExoI, and the 5′ ends are recessed in the presence of Lambda exonuclease.

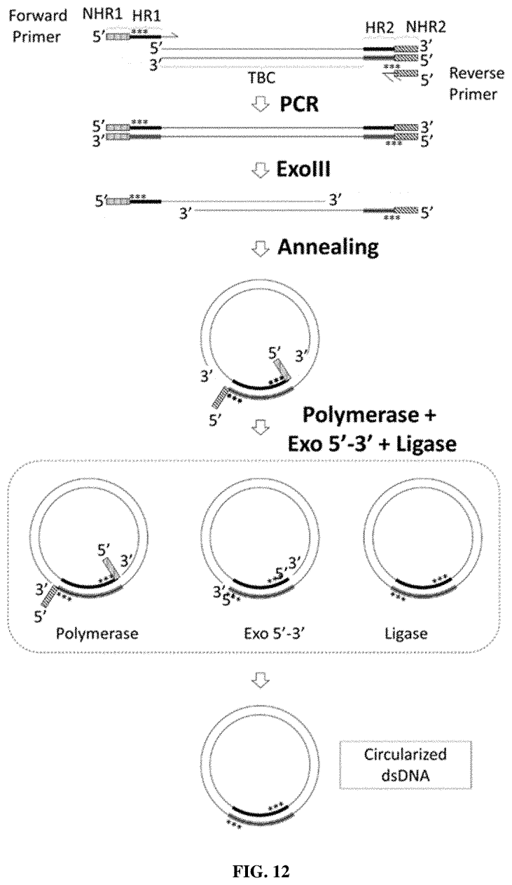

As shown in FIG. 12, the presence of thiophosphate nucleotide analogs does not interfere in the recessing of 3′ ends by ExoIII.

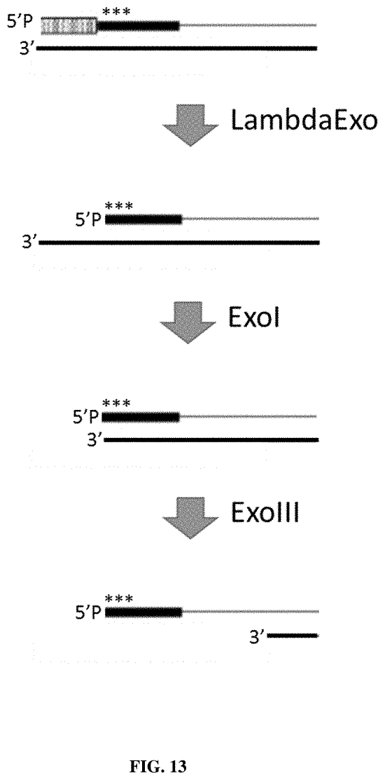

As shown in FIG. 13, Lambda exonuclease recesses 5′ ends up to the thiophosphate nucleotide analog, where it stops recessing. ExoI recesses 3′ ends until reaching the 5′ phosphate on the other strand and stops recessing, so as to form blunt ends of each ends of the linear nucleic acid. Finally, ExoIII recesses the 3′ ends.

Example 10: Circularization of Linear Double Stranded DNA

a) Experimental Design

A linear DNA for circularization is obtained by enzymatic cleavage of a cloned plasmid. The plasmid (6,711 bp) is cut at one StuI site and two BsrBI sites providing three fragments: 4,528 bp of a sequence to be circularized, 1,901 bp and 282 bp of bacterial backbone. The fragment 4,528 bp obtained by cleavage at StuI and BsrBI sites contains the sequence CCTGTGTGAAATTGTTATCCG (SEQ ID NO: 45) repeated on its both 5′ terminus and 3′ terminus. The plasmid was treated with StuI and BsrBI restriction enzymes in CutSmart® buffer from New England Biolabs® following standard protocol. The purified DNA (final concentration 30 ng/μL in 10 μL) was mixed with a buffer containing Tris-Acetate pH 8.0 73 mM, Potassium Acetate 60 mM, different Magnesium Acetate concentrations from 0 mM to 26 mM. Next, 20u of Exonuclease III in 2 μL the same buffer was added and incubated as following: 30° C. for 2 mM, 75° C. for 10 mM, 60° C. for 5 mM, 4° C. for 10 mM, successively. The product of the reaction was loaded on an agarose gel for electrophoresis.

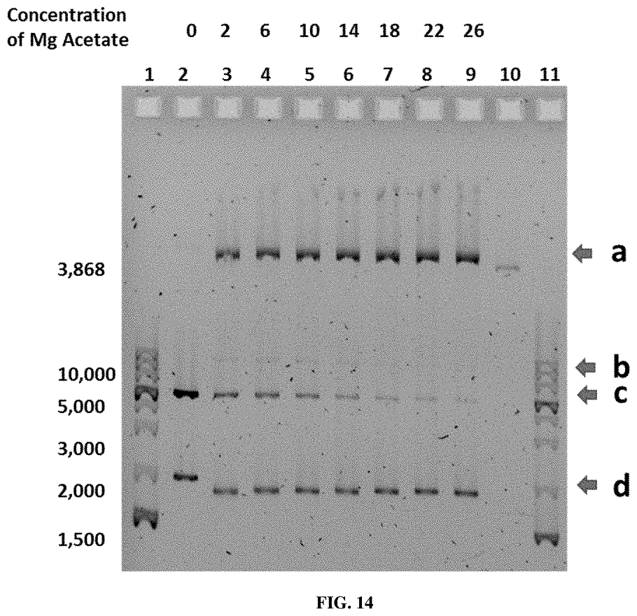

b) Results (FIG. 14)

As shown in FIG. 14, high concentration of divalent cations, such as Magnesium acetate, leads to more efficient production of circularized DNA. When the intensities of bands that correspond to circularized 4,528 bp, linear 4,528 bp fragment and 1,901 bp fragment were compared at different magnesium concentrations, it was observed a gradual intensity decrease of linear 4,528 bp fragment, which becomes practically invisible at 26 mM concentration while the bands of circularized 4,528 bp gradually become stronger at higher magnesium concentrations and reach the highest intensity at 26 mM magnesium concentration (lane 9). In addition, the circularized relaxed DNA have very similar localization as compared to the molecular weight marker (lane 10) that corresponds to the relaxed plasmid DNA 3,868 bp obtained by treatment with the nicking enzyme Nt.BbvCI.

### Example 11: Efficiency of the Circularization Method at High Concentration of DNA Molecules

a) Experimental Design

The plasmid was treated with StuI (MbiI) and BsrBI (Eco147I) restriction enzymes in Anza® buffer from Thermo Fisher Scientific® following standard protocol. The purified DNA molecules (final concentration 30 ng/μL in 10 μL or 150 ng/μL in 50 μL) were mixed with a buffer containing Tris-Acetate pH 8.0 73 mM, Potassium Acetate 60 mM, Magnesium Acetate 30 mM. Next, the Exonuclease III was added either 20u in 2 μL buffer for 30 ng/μL specimen or 0.5 μL of stock solution 200u/μL for 150 ng/μL samples. The reaction was conducted as following: 30° C. for 2 mM, 75° C. for 10 mM, 60° C. for 5 min, 4° C. for 10 min, successively. The same amount of the reaction product was loaded on agarose gel for electrophoresis.

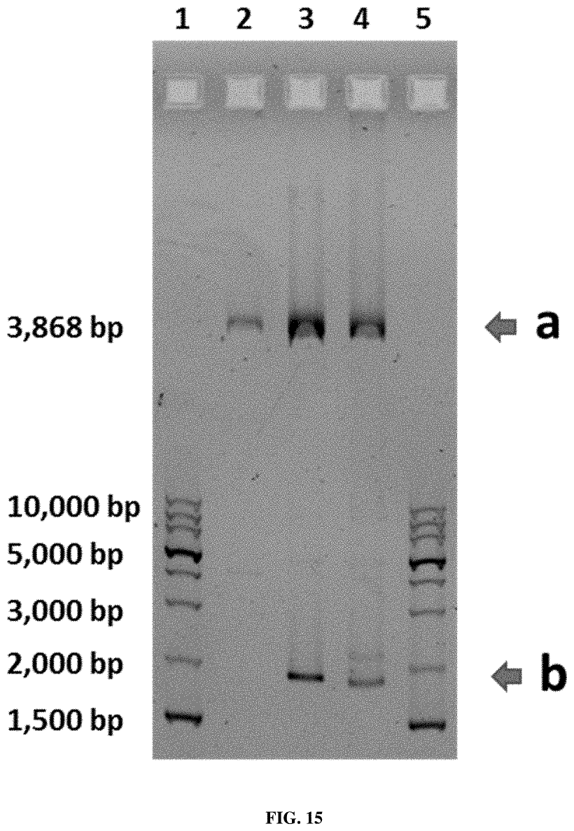

b) Results (FIG. 15)

As shown in FIG. 15, the circularization method according to the invention provides high yields of circularized DNA molecules at increased DNA concentration, as indicated by the presence of two major bands: the circularized 4,528 bp and linear 1,901 bp both at expected molecular weight position. The linear 4,528 bp band and concatemers are not visible. These results show the scalability of the DNA circularization.

### Example 12

a) Experimental Design

The DNA molecules for transfection were circular 4,528 bp DNA molecules obtained with the use of the method according to the invention and the 6,711 bp plasmid DNA molecules, the parental DNA for production of circular 4,528 bp DNA molecules (see Example 10). Both DNA molecules contain a genetic construct for eukaryotic GFP gene expression. The cells were passaged into 24-well plate at 40% to 70% confluency in standard conditions. The transfection reagent JetOptimus® (Polyplus® transfection) was applied with four amounts of DNA 1,000 ng, 750 ng, 500 ng or 250 ng following the provided protocol. The cells were analyzed under the fluorescent microscope 24 hours after the cell transfection.

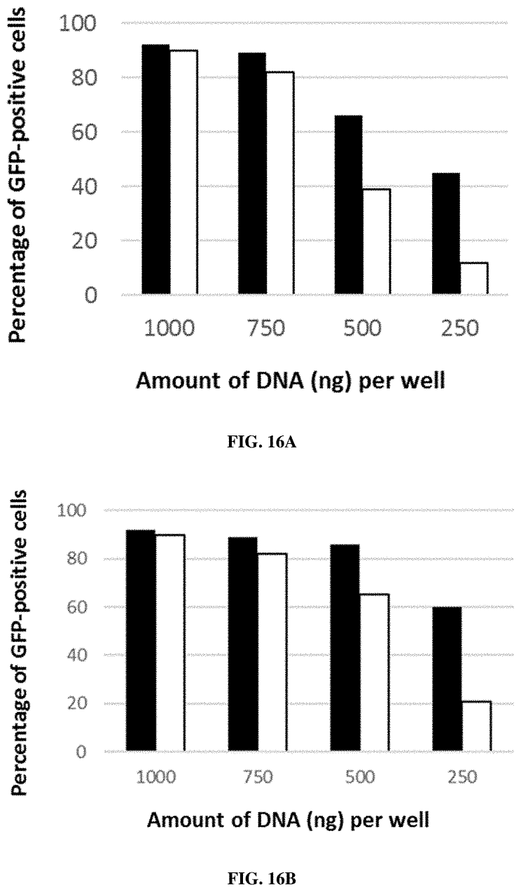

b) Results (FIG. 16A-B)

As shown in FIG. 16A-B, there was obtained advanced transfection efficiency with the product of the circularization method. These results show that the circularization method according to the invention allows providing high percentages of transfected cells even at low DNA amounts per well.

1-15. (canceled)

16. A method for the circularization of a double stranded DNA nucleic acid, said method comprising the steps of:
a) providing a linear or circular double stranded DNA nucleic acid comprising a nucleic acid of the following formula (I):
NHR1-SOR1x-HR1-TBC-HR2-SOR2y-NHR2 (I),
wherein:
TBC represents a core double stranded DNA nucleic acid to be circularized;
HR1 and HR2 represent identical homologous double stranded DNA nucleic acids of identical orientation;
SOR1x and SOR2y represent nucleic acids having a length of about 5 bp to about 60 bp comprising a site of restriction capable of generating 3′ overhangs having a length of at least 4 nucleotides and having as 3′ terminal nucleotide A, T or G; and x and y are 0 or 1; when x and y are 1, TBC further comprises 2 sites of nicking restriction on different DNA strands of the formula (I) each located about 0 nucleotide to about 100 nucleotides respectively from the 3′ end of HR1 and from the 3′ end of HR2;
NHR1 and NHR2 represent distinct non-homologous double stranded DNA nucleic acids having a length of 0 bp to about 200 bp;
b1) when x and y are 0, digesting the circular double stranded DNA nucleic acid comprising a nucleic acid of formula (I) from step a), in the presence of a restriction enzyme that cleaves a restriction site that is not located in any one of HR1, HR2 and TBC, so as to obtain a linearized nucleic acid;
b2) when x and y are 1,
optionally digesting the circular double stranded DNA nucleic acid comprising a nucleic acid of formula (I) from step a), in the presence of a restriction enzyme that cleaves a restriction site that is not located in any one of HR1, HR2 and TBC, so that to generate a linearized nucleic acid;
digesting the circular, linear or linearized nucleic acid in the presence of (i) a polypeptide with nickase activity capable of introducing a nick within TBC, and (ii) restriction enzyme(s) capable of digesting the corresponding site of restriction in SOR1 and SOR2;
c) recessing both ends of identical orientation of the nucleic acid obtained at step b1) or step b2), in the presence of a polypeptide having a 3′-5′ nuclease activity, so that HR1 and HR2 are capable of forming overlapping overhangs;
d) annealing the DNA nucleic acid obtained at step c), thereby generating 2 gaps, 2 nicks, or 1 nick and 1 gap;
e) filling the 1 or 2 gaps generated at step d), step e) being optional when 2 nicks are generated at step d);
f) optionally removing the NHR1 and NHR2 at both 5′ ends, when the length of the NHR1 and NHR2 is superior to 0 bp; and
g) sealing at least one nick, so as to obtain a circularized double stranded DNA nucleic acid, in which at least one strand is continuous.

17. The method according to claim 16, wherein step b) or step c) is preceded by, or concomitantly performed with, a step comprising incubating the linear double stranded DNA nucleic acid of step a) with an alkaline phosphatase and/or a polypeptide with a type I exonuclease activity.

18. The method according to claim 16, wherein step d) is performed in the presence of divalent and/or trivalent cations.

19. The method according to claim 16, wherein step e) is performed in the presence of a DNA polymerase with no strand displacement activity and with no 5′ to 3′ exonuclease activity and dNTPs and/or of oligonucleotides with 5′-phosphate having a length of about 8 nucleotides to about 100 nucleotides and being complementary to the nucleic acids of the gaps, provided the polypeptide having a 3′-5′ nuclease activity has been inactivated.

20. The method according to claim 16, wherein step f) is performed in the presence of a polypeptide having a 5′-3′ nuclease activity.

21. The method according to claim 16, wherein step f) is performed in the presence of Exonuclease VII (Exo VII), RecJ, or Flap endonuclease 1 (FEN1).

22. The method according to claim 16, wherein step g) is performed in the presence of a mesophilic DNA ligase.

23. The method according to claim 16, wherein step g) is performed in the presence of a T4 DNA ligase.

24. The method according to claim 16, wherein said method further includes a step of binding the double stranded DNA nucleic acid to one or more non-nucleic acid moiety (moieties), so as to generate a functionalized binding of the double stranded DNA nucleic acid.

25. The method according to claim 24, wherein said double stranded DNA nucleic acid is a functionalized circularized double stranded DNA nucleic acid.

26. The method according to claim 24, wherein said one or more non-nucleic acid moiety (moieties) is selected in the group comprising linkers, polypeptides, particles, surfaces, and combinations thereof.

27. A circularized double stranded DNA nucleic acid obtainable by a method according to claim 16.

28. The circularized double stranded DNA nucleic acid according to claim 27, wherein said nucleic acid is functionalized and/or relaxed.

29. A host cell comprising a circularized double stranded DNA nucleic acid obtainable by a method according to claim 16.

30. A method for implementing gene therapy, and/or DNA vaccination, and/or cell therapy, and/or genome editing, and/or production of induced pluripotent stem cells, and/or transfection or transformation of cultured cells; or for the storage of data, and/or for the sequencing of nucleic acids, and/or for the production of rolling circle DNA, and/or for the production of proteins, and/or for the production of RNA, and/or in metabolic pathway engineering, and/or in molecular biology, and/or for the transformation of bacteria, and/or for the production of viruses, said method comprising at least the following steps:
a) providing a linear or circular double stranded DNA nucleic acid comprising a nucleic acid of the following formula (I):
NHR1-SOR1x-HR1-TBC-HR2-SOR2y-NHR2 (I),
wherein:
TBC represents a core double stranded DNA nucleic acid to be circularized;
HR1 and HR2 represent identical homologous double stranded DNA nucleic acids of identical orientation;
SOR1x and SOR2y represent nucleic acids having a length of about 5 bp to about 60 bp comprising a site of restriction capable of generating 3′ overhangs having a length of at least 4 nucleotides and having as 3′ terminal nucleotide A, T or G; and x and y are 0 or 1; when x and y are 1, TBC further comprises 2 sites of nicking restriction on different DNA strands of the formula (I) each located about 0 nucleotide to about 100 nucleotides respectively from the 3′ end of HR1 and from the 3′ end of HR2;
NHR1 and NHR2 represent distinct non-homologous double stranded DNA nucleic acids having a length of 0 bp to about 200 bp;
b1) when x and y are 0, digesting the circular double stranded DNA nucleic acid comprising a nucleic acid of formula (I) from step a), in the presence of a restriction enzyme that cleaves a restriction site that is not located in any one of HR1, HR2 and TBC, so as to obtain a linearized nucleic acid;
b2) when x and y are 1,
optionally digesting the circular double stranded DNA nucleic acid comprising a nucleic acid of formula (I) from step a), in the presence of a restriction enzyme that cleaves a restriction site that is not located in any one of HR1, HR2 and TBC, so that to generate a linearized nucleic acid;
digesting the circular, linear or linearized nucleic acid in the presence of (i) a polypeptide with nickase activity capable of introducing a nick within TBC, and (ii) restriction enzyme(s) capable of digesting the corresponding site of restriction in SOR1 and SOR2;
c) recessing both ends of identical orientation of the nucleic acid obtained at step b1) or step b2), in the presence of a polypeptide having a 3′-5′ nuclease activity, so that HR1 and HR2 are capable of forming overlapping overhangs;
d) annealing the DNA nucleic acid obtained at step c), thereby generating 2 gaps, 2 nicks, or 1 nick and 1 gap;
e) filling the 1 or 2 gaps generated at step d), step e) being optional when 2 nicks are generated at step d);
f) optionally removing the NHR1 and NHR2 at both 5′ ends, when the length of the NHR1 and NHR2 is superior to 0 bp; and
g) sealing at least one nick, so as to obtain a circularized double stranded DNA nucleic acid, in which at least one strand is continuous.

31. A kit for the circularization of a double stranded DNA nucleic acid, said kit comprising:
a) a polypeptide having a 3′-5′ nuclease activity;
b) a polypeptide having a 5′-3′ nuclease activity;
c) a DNA polymerase;
d) a mesophilic DNA ligase; and optionally,
e) one or more buffer(s).

32. The kit according to claim 31, wherein said kit comprises at least two vials:
the first vial comprising the polypeptide having a 3′-5′ nuclease activity; and optionally alkaline phosphatase and/or a polypeptide with a type I exonuclease activity;
the second vial comprising a DNA ligase; and optionally a polypeptide having a 5′-3′ nuclease activity, and/or a DNA polymerase with no strand displacement activity and with no 5′ to 3′ exonuclease activity, ATP and dNTPs.

33. The kit according to claim 31, wherein said kit further comprises one or more primer(s) selected in the group consisting of a primer of sequence SEQ ID NO. 43 and a primer of sequence SEQ ID NO. 44.
